(19) United States
(12) Patent Application Publication
(10) Pub. No.: US 2012/0164653 A1
(43) Pub. Date: Jun. 28, 2012

(54) METHODS FOR THE DIAGNOSIS OF MULTIPLE SCLEROSIS BASED ON ITS MICRORNA EXPRESSION PROFILING

(76) Inventors: David Otaeguibichot, Begiristain (ES); Adolfo López De Munain Arregl, Begiristain (ES); Boria Calvo Molinos, Leioa (ES); Rubén Armañanzas Arnedillo, Boadilla del Monte (ES); Iñaki Inza Cano, Leioa (ES); José Antonio Lozano Alonso, Leioa (ES)

(21) Appl. No.: 13/382,793

(22) PCT Filed: Jul. 8, 2010
(86) PCT No.: PCT/EP2010/059844

§ 371 Date: Mar. 15, 2012

(30) Foreign Application Priority Data
Jul. 8, 2009 (EP) 09382108.0

(51) Int. Cl. C12Q 1/68

(52) U.S. Cl. 435/6.12

(57) ABSTRACT

The invention relates, in general, to a method for the diagnosis of multiple sclerosis based on determining amounts of one or more micro-RNAs correlated with multiple sclerosis in a biological sample from a subject.

### FIELD OF THE INVENTION

The invention relates, in general, to methods for diagnosis of multiple sclerosis. In particular, the invention relates to diagnostic methods based on determining amounts of one or more micro-RNAs correlated with multiple sclerosis in a biological sample from a subject.

### BACKGROUND OF THE INVENTION

Multiple sclerosis (MS) is an autoimmune demyelinating disease of the central nervous system (CNS). It begins most commonly during late adolescence, young adulthood, or mid-life, and it is one of the most incapacitating diseases in this age range.

MS causes attacks of neurological dysfunction (loss of vision, difficulty in walking or moving a limb, vertigo, loss of sensation) or progressive dysfunction in these same areas. These “attacks”, also known as relapses, typically last for a few days, and resolve spontaneously. However, patients may not always completely recover from an attack and are sometimes left with a disability. Although most patients experience attacks with little or no progressive disability, approximately 10-15% have progressive symptoms from onset, called primary progressive forms. Furthermore, more than 80% of patients will ultimately develop progressive symptoms after a prolonged period of exacerbations, usually after 10-20 years.

Etiologically, MS is a complex disease in which both genetic and environmental factors play a role. The genetics of MS are also complex without a clear inheritance pattern. The most relevant candidate genomic region is the HLA system [Oksenberg, J. R. & Barcellos, L. F. Multiple sclerosis genetics: leaving no stone unturned. Genes Immun. 6, 375-387 (2005)], although several other genes are currently being described as important risk factors involved in MS, as for example IL2RA [Alcina, A. et al. IL2RA/CD25 gene polymorphisms: uneven association with multiple sclerosis (MS) and type 1 diabetes (T1D). PLoS. ONE. 4, e4137 (2009)] or IL7R genes [Gregory, S. G. et al. Interleukin 7 receptor alpha chain (IL7R) shows allelic and functional association with multiple sclerosis. Nat. Genet 39, 1083-1091 (2007)].

Gene expression profiling has been a useful tool to provide information about the molecular pathways involved in MS pathogenesis [Achiron, A., Gurevich, M., Friedman, N., Kaminski, N., & Mandel, M. Blood transcriptional signatures of multiple sclerosis: unique gene expression of disease activity. Ann. Neurol. 55, 410-417 (2004); Baranzini, S. E. et al. Transcription-based prediction of response to IFNbeta using supervised computational methods. PLoS. Biol. 3, e2 (2005); Ramanathan, M. et al. In vivo gene expression revealed by cDNA arrays: the pattern in relapsing-remitting multiple sclerosis patients compared with normal subjects. J. Neuroimmunol. 116, 213-219 (2001)]. Several new studies have identified different expression patterns between relapses and remission [Otaegui, D. et al. Increased transcriptional activity of milk-related genes following the active phase of experimental autoimmune encephalomyelitis and multiple sclerosis. J. Immunol. 179, 4074-4082 (2007); Satoh, J., Misawa, T., Tabunoki, H., & Yamamura, T. Molecular network analysis of T-cell transcriptome suggests aberrant regulation of gene expression by NF-kappaB as a biomarker for relapse of multiple sclerosis. Dis. Markers 25, 27-35 (2008)] showing that this clinical differentiation of two states of the disease also has a molecular correlation.

Recently a new expression and protein synthesis modulator has been identified: the small non-coding RNA molecules (microRNA or miRNA). These miRNA are single-stranded RNA molecules of about 20-25 nucleotides (nt) encoded by nuclear genes (70-150 nt) and highly conserved among species. These genes are not translated into proteins but are processed from primary transcripts (called pri-miRNA) to short stem-loop structures called pre-miRNA and finally to functional miRNA. The expression pattern of miRNA varies over time and between tissues. These mature miRNA molecules are partially complementary to one or more mRNA sequences and they function through sequence-specific down-regulation of their target mRNA via mRNA degradation or inhibition of translation [Bartel, D. P. MicroRNAs: genomics, biogenesis, mechanism, and function. Cell 116, 281-297 (2004)]. Initial estimates suggested there were more than 500 validated human miRNA [Griffiths-Jones, S., et al. miRBase: microRNA sequences, targets and gene nomenclature. Nucleic Acids Res. 34, D140-D144 (2006); Griffiths-Jones, S. The microRNA Registry. Nucleic Acids Res. 32, D109-D111 (2004)], although in the public database around 700 were proposed in October 2008 (www.microrna.sangerac.uk).

It has been predicted that miRNA may regulate around 30% of all cellular mRNA so they should play a critical role in virtually all cellular functions [Lewis, B. P., Burge, C. B., & Bartel, D. P. Conserved seed pairing, often flanked by adenosines, indicates that thousands of human genes are microRNA targets. Cell 120, 15-20 (2005)].

Although misregulation of miRNA expression has been characterized mostly in cancer, it has recently been studied in many other diseases. Thus, miRNA has been proposed as a regulator of immune cell development [Baltimore. D., Boldin, M. P., O'Connell, R. M., Rao, D. S., & Taganov, K. D. MicroRNAs: new regulators of immune cell development and function. Nat. Immunol. 9, 839-845 (2008)], playing a role in the inflammatory response [O'Connell, R. M., Taganov, K. D. Boldin, M. P., Cheng, G., & Baltimore, D. MicroRNA-155 is induced during the macrophage inflammatory response. Proc. Natl. Acad. Sci U.S.A. 104, 1604-1609 (2007)], and as a key player in the pathogenesis of neurodegenerative diseases [Nelson, P. T., Wang, W. X., & Rajeev, B. W. MicroRNAs (miRNAs) in neurodegenerative diseases. Brain Pathol. 18, 130-138 (2008)]. Further, WO 2008/147974 discloses the role of some miRNAs (e.g., hsa-mir-493) in the pathogenesis of asthma; WO 2009/009457 discloses methods of diagnosis and prognosis of Alzheimer's disease in subjects by measuring amounts of one or more miRNAs (e.g., hsa-mir-186 and hsa-mir-493-3p); WO 2008/153692 discloses the role of hsa-mir-186 and hsa-mir-96 in brain tumors and hsa-mir-30a-5p is up-regulated by wt-p53 in human colon cancer cell lines HCT-116 (wt-p53) [Yaguang X et al., Clin. Cancer Res 2006; 12(7):2014-2024].

Currently MS is diagnosed by clinical observation following the Polman Criteria [Polman C H, Reingold S C, Edan G, Filippi M, Hartung H P, Kappos L. Lublin F D, Metz L M, McFarland H F, O'Connor P W, Sandberg-Wollheim M, Thompson A J, Weinshenker B G, Wolinsky J S. Diagnostic criteria for multiple sclerosis: 2005 revisions to the “McDonald Criteria Ann Neurol. 2005 December; 58(6):840-6]. Basically these criteria are focussed in the dissemination of lesions in both time and space combining with other clinical and paraclinical diagnostic methods. Time is one of the drawbacks of said criteria since it is one of the parameters used in the diagnosis and a patient could need a period between six months to one year to be diagnosed with MS. In this scenario an objective tool that could help in the diagnosis of MS would be very useful, reducing the time, and, maybe, the cost of the diagnostic.

On the other hand, the definition of relapse is some times difficult because some relapses, i.e. sensorial symptoms, could have no neurological origin. Moreover, some relapses are subclinical so they are manifested only in the magnetic resonance imaging (MRI). Thus, a biological marker of the relapse could be useful not only to characterize the relapse but also to evaluate the treatment.

### SUMMARY OF THE INVENTION

The present invention is based upon the discovery that some miRNAs are expressed at different levels not only in subjects afflicted with MS (i.e., MS patients) versus controls but also in the relapse status of the disease. These observations provide a basis for the concept that assays of microRNA levels may be used in diagnosing MS and in the post-therapy monitoring of patients. In particular, a comparison can be made between the levels of miRNA of a test subject and in that of one or more control subjects. Comparisons can be made directly. Measurements of miRNA levels may be carried out using singleplex (involving one set of primers) or multiplex (involving more than one set of primers) qRT-PCR.

Inventors have studied the expression pattern of 364 miRNAs in peripheral blood mononuclear cells (PBMC) obtained from multiple sclerosis patients in relapse status, in remitting status and healthy controls with the aim of understanding the regulatory mechanisms of said stages. Expression values were obtained by means of qPCR. The most relevant miRNA were validated in an independent set of samples. In order to determine the effect of the miRNA pattern, the expression of some predicted target genes was studied by qPCR. Gene interaction networks were constructed in order to obtain a system biology and multivariate view of the experimental data. The data analysis and later validation reveal that some miRNAs may be relevant for the diagnosis of MS, or at the time of relapse or may be involved in the remitting phenomena (Examples 1 and 2).

The different aspects of the invention are defined in the attached claims and disclosed in detail in the description of the invention.

### BRIEF DESCRIPTION OF DRAWINGS

FIG. 1 is a diagram depicting the gene interaction networks from the qPCR data. A: Relapse status versus remitting status. B: Remitting status versus controls.

FIG. 2 are graphs showing data the percentage of the targets founded in the EAE experiment (A: EAE lymph node target genes; B: EAE spinal cord target genes). The found targets were grouped in up-regulated, down-regulated and equally regulated between the EAE model and the control. The data from the 8 selected miRNAs are shown (clear) as well as the data from the randomly selected 11 miRNAs (dark).

FIG. 3 is a graph showing different comparisons made in the data analysis. Inventors compared all patients, relapse and remission group with controls (which was the reference group) and relapse with the remission group (taking remission as the reference group).

FIG. 4 is a bar diagram showing the relative quantification of 14 miRNAs making 4 comparisons: relapse vs control, remission vs control, relapse vs remission and MS vs control, Stars indicate that RQ values obtained are statistically significant with Benjamini-Hochberg corrected p-value<0.05.

FIG. 5 is a boxplot of the average SU metric values for each miRNA in the study (Example 2). Values were computed using a 10 times 10 cross-validation scheme. The boxplot shows the average values and the deviation of those values.

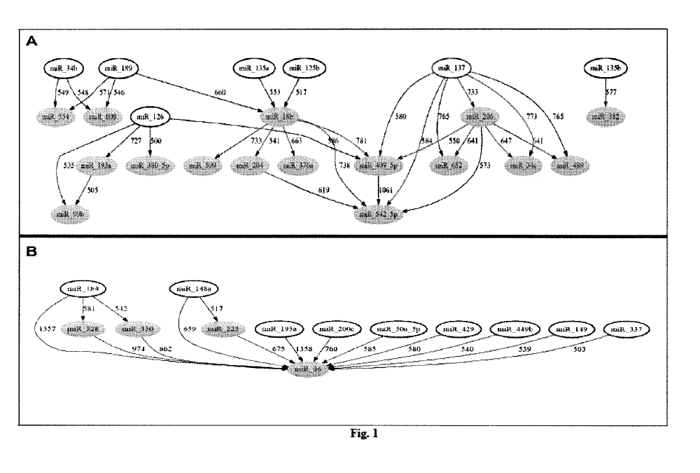

### DETAILED DESCRIPTION OF THE INVENTION

### Definitions

A “microRNA” (“miRNA”) is a naturally occurring, small non-coding RNA that is about 17 to about 25 nucleotide (nt) in length in its biologically active form that negatively regulates mRNA translation on a sequence-specific manner. MicroRNAs (miRNAs) post-transcriptionally regulate gene expression by repressing target mRNA translation. It is thought that miRNAs function as negative regulators, i.e. greater amounts of a specific miRNA will correlate with lower levels of target gene expression. There are three forms of miRNAs existing in vivo, primary miRNAs (pri-miRNAs), premature miRNAs (pre-miRNAs), and mature miRNAs. Primary miRNAs (pri-miRNAs) are expressed as stem-loop structured transcripts of about a few hundred bases to over 1 kilobase (kb). The pri-miRNA transcripts are cleaved in the nucleus by an RNase II endonuclease that cleaves both strands of the stem near the base of the stem loop. The cleavage product, the premature miRNA (pre-miRNA) is about 60 to about 110 nt long with a hairpin structure formed in a fold-back manner. Pre-miRNA is transported from the nucleus to the cytoplasm. Pre-miRNAs are processed further in the cytoplasm by another RNase II endonuclease to form miRNA duplexes. The miRNA duplex binds to the RNA-induced silencing complex (RISC), where the antisense strand is preferentially degraded and the sense strand mature miRNA directs RISC to its target site. It is the mature miRNA that is the biologically active form of the miRNA. Identified miRNAs are registered in the miRNA database miRBase (http://microrna.sanger.ac.uk/). It is believed that the number of miRNAs could exceed 1,000, which constitute approximately 1-5% of the expressed genes. Bioinformatics analyses suggest that, on average, each miRNA may regulate about 200 genes. Therefore, as much as 30% to 50% of all human genes may be under miRNA control. Since miRNAs may play important roles in cell development, proliferation and apoptosis, their expression patterns are highly regulated; in fact, deregulation of miRNA function might contribute to many human diseases.

A “sample”, as defined herein, is a small part of a subject, representative of the whole and may be constituted by a biopsy or a body fluid sample. Biopsies are small pieces of tissue and may be fresh, frozen or fixed, such as formalin-fixed and paraffin embedded (FFPE). Body fluid samples may be blood, plasma, serum, urine, sputum, cerebrospinal fluid, milk, or ductal fluid samples and may likewise be fresh, frozen or fixed. Samples may be removed surgically, by extraction i.e. by hypodermic or other types of needles, by microdissection or laser capture. The sample should contain any biological material suitable for detecting the desired biomarker (miRNA), thus, said sample should, advantageously comprise cell material from the subject. Thus, in a particular embodiment, the sample is a body fluid sample such as a blood sample; preferably, the sample comprises peripheral blood mononuclear cells (PB MC) from the subject.

A “reference sample”, as used herein, means a sample obtained from subjects, preferably two or more subjects, known to be free of the disease (MS) or, alternatively, from the general population. The suitable reference expression levels of miRNAs can be determined by measuring the expression levels of said miRNAs in several suitable subjects, and such reference levels can be adjusted to specific subject populations. In a preferred embodiment, the reference sample is obtained from a pool of healthy subjects or from subjects without prior history of MS. The expression profile of the miRNAs in the reference sample can, preferably, be generated from a population of two or more subjects; for example, the population can comprise 3, 4, 5, 10, 15, 20, 30, 40, 50 or more subjects.

A “subject”, as used herein, refers to a mammal, human or non-human, under observation, preferably a human being. The subject may be any subject, a subject predisposed of a disease (e.g., MS) or a subject suffering from said disease.

“Multiple sclerosis” (“MS”) is a disease in which there are demyelination foci of several sizes throughout the white matter of the central nervous system (CNS), sometimes extending into the gray matter, giving rise weakness, incoordination, paresthesias, speech disorders, and visual complaints. MS is a disease of unknown etiology with a prolonged course involving many remissions and relapses. The term “multiple sclerosis” or “MS”, as used herein, is meant to include benign multiple sclerosis (benign MS), relapsing-remitting multiple sclerosis (RRMS), secondary progressive multiple sclerosis (SPMS), primary progressive multiple sclerosis (PPMS), and progressive-relapsing multiple sclerosis (PRMS). These subtypes or forms of the disease (MS) may be distinguished from one another on the basis of the course of the disease, of the type of inflammation involved, and through the use of magnetic resonance imaging (MRI). Chronic progressive multiple sclerosis is a term used to collectively refer to SPMS, PPMS, and PRMS. The relapsing forms of multiple sclerosis are SPMS with superimposed relapses, RRMS, and PRMS.

As used herein, the expression “diagnosis” or “diagnosing” relates to methods by which the skilled artisan can estimate and even determine whether or not a subject is suffering from a given disease or condition. The skilled artisan often makes a diagnosis on the basis of one or more diagnostic indicators, such as for example a biomarker (e.g., a miRNA or a set of miRNAs), the amount (including presence or absence) of which is indicative of the presence, severity, or absence of the condition. It also relates to evaluating the probability according to which a subject suffers from a disease. As it will be understood by persons skilled in the art, such evaluation, although it is preferred that it is, normally may not be correct for 100% of the subjects to be diagnosed. The term, however, requires that a statistically significant part of the subjects can be identified as suffering from the disease or having a predisposition for it. The person skilled in the art can determine if a part is statistically significant without further delay by using several well known statistic evaluation tools, for example, determination of confidence intervals, determination of the p value, Student's t-test, Mann-Whitney test, etc. The details are in Dowdy and Wearden, Statistics for Research, John Wiley & Sons, New York 1983. The preferred confidence intervals are of at least 50%, at least 60%, at least 70%, at least 80%, at least 90%, at least 95%. The p values are preferably 0.2, OA, 0.05.

Along with diagnosis, clinical disease prognosis is also an area of great concern and interest. It is important to know the stage and rapidity of advancement of the MS in order to plan the most effective therapy. If a more accurate prognosis can be made, appropriate therapy, and in some instances less severe therapy for the patient can be chosen. Measurement of miRNA levels disclosed herein can be useful in order to categorize subjects according to advancement of MS who will benefit from particular therapies and differentiate from other subjects where alternative or additional therapies can be more appropriate.

Further, the expression “method of diagnosing” as used herein relates to a method that may essentially consist of the steps mentioned below (or may include additional steps. However, it must be understood that the method, in a preferred embodiment, is a method that is carried out in vitro, i.e., it is not carried out in the human or animal body.

The terms “PCR reaction”, “PCR amplification”, “PCR”, “pre-PCR”, “Q-PCR”, “qPCR”, “real-time quantitative PCR” and “real-time quantitative RT-PCR” are interchangeable terms used to mean the use of a nucleic acid amplification system, which multiplies the target nucleic acids being detected. Examples of such systems include the polymerase chain reaction (PCR) system and the ligase chain reaction (LCR) system. Other methods recently described and known to the person skilled in the art include the nucleic acid sequence based amplification and Q Beta Replicase systems. The products formed by said amplification reaction may or may not be monitored in real time or only after the reaction as an end-point measurement.

### Diagnostic Methods of the Invention

The inventors have identified a set of specific miRNA which are differentially expressed in MS patients with respect to reference (non-MS) samples.

Accordingly, in an aspect, the invention relates to a method of diagnosing Multiple Sclerosis (MS) in a subject, hereinafter referred to as the method of the invention, which comprises comparing:
a) the level of expression of a miRNA selected from the group consisting of hsa-mir-18b, hsa-mir-493, hsa-mir-599, hsa-mir-96, hsa-mir-148a, hsa-mir-184, hsa-mir-193, hsa-mir-193a, hsa-mir-328, hsa-mir-409-5p, hsa-mir-449b, hsa-mir-485-3p, hsa-mir-200c, hsa-mir-330, hsa-mir-554, hsa-mir-137 and combinations thereof, in a sample from said subject; and
b) the normal level of expression of said miRNA(s) in a reference sample,

wherein a statistically significant deregulation in the level of expression of said miRNA hsa-mir-18b, hsa-mir-493, hsa-mir-599, hsa-mir-96, hsa-mir-148a, hsa-mir-184, hsa-mir-193, hsa-mir-193a, hsa-mir-328, hsa-mir-409-5p, hsa-mir-449b, hsa-mir-485-3p, hsa-mir-200c, hsa-mir-330, hsa-mir-554 or hsa-mir-137, in the subject sample with respect to the normal level of said miRNA(s) in the reference sample is indicative that the subject is afflicted with MS.

Briefly, the method of the invention involves obtaining a sample from the subject (i.e., a test sample) and assaying said sample to determine the level of expression (i.e., the concentration or amount) of one or more miRNAs selected from the group consisting of hsa-mir-18b, hsa-mir-493, hsa-mir-599, hsa-mir-96, hsa-mir-148a, hsa-mir-184, hsa-mir-30a-5p, hsa-mir-193, hsa-mir-193a, hsa-mir-328, hsa-mir-409-5p, hsa-mir-449b, hsa-mir-485-3p, hsa-mir-200c, hsa-mir-330, hsa-mir-554, hsa-mir-137 and combinations thereof. The expression level of said miRNAs can be determined by any suitable method, e.g., multiplex and/or singleplex real-time RT-PCR, including quantitative real time reverse transcriptase PCR (qRT-PCR); single-molecule detection; bead-based flow cytometric methods; assays using arrays of nucleic acids; etc.

The sample can be obtained by any conventional method depending on the sample to be analyzed. In a particular embodiment, the sample is a body fluid sample such as a blood sample, e.g., a sample comprising PBMC from the subject. Before analyzing the sample, it will often be desirable to perform one or more sample preparation operations upon the sample. Typically, these sample preparation operations include manipulations such as concentration, suspension, extraction of intracellular material, e.g., nucleic acids from tissue/whole cell samples and the like, amplification of nucleic acids, fragmentation, transcription, labeling and/or extension reactions. Nucleic acids, especially RNA and specifically miRNA can be isolated using any techniques known in the art. There are two main methods for isolating RNA: (i) phenol-based extraction and (ii) silica matrix or glass fiber filter (GFF)-based binding. Phenol-based reagents contain a combination of denaturants and RNase inhibitors for cell and tissue disruption and subsequent separation of RNA from contaminants. Phenol-based isolation procedures can recover RNA species in the 10-200-nucleotide range e.g., miRNAs, 5S rRNA, 5.8S rRNA, and U1 snRNA. If a sample of “total” RNA was purified by the popular silica matrix column or OFF procedure, it may be depleted in small RNAs. Extraction procedures such as those using Trizol or TriReagent, however will purify all RNAs, large and small, and are the recommended methods for isolating total RNA from biological samples that will contain miRNAs/siRNAs. Any method required for the processing of a sample prior to quantifying the level of the miRNAs according to the method of the invention falls within the scope of the present invention. These methods are typically well known by a person skilled in the art.

According to the method of the invention, the specific miRNAs that are tested for include one or more of the following: hsa-mir-18b, hsa-mir-493, hsa-mir-599, hsa-mir-96, hsa-mir-148a, hsa-mir-184, hsa-mir-30a-5p, hsa-mir-193, hsa-mir-193a, hsa-mir-328, hsa-mir-409-5p, hsa-mir-449b, hsa-mir-485-3p, hsa-mir-200c, hsa-mir-330, hsa-mir-554, and hsa-mir-137. The designations provided are standard in the art and are associated with specific sequences that can be found at the miRNA registry (http://microma.sanger.ac.uk/sequences/). In all cases, they refer to human sequences as shown in Table 1.

TABLE 1
MicroRNA sequences

| | | SEQ |
|---|---|---|
| MicroRNA | Sequence | ID NO: |
| hsa-miR-18b | UAAGGUGCAUCUAGUGCAGUUA | 1 |
| hsa-mir-493 | UUGUACAUGGUAGGCUUUCAUU | 2 |
| hsa-mir-599 | GUUGUGUCAGUUUAUCAAAC | 3 |
| hsa-mir-96 | UUUGGCACUAGCACAUUUUUGC | 4 |
| hsa-mir-148a | UCAGUGCACUACAGAACUUUGU | 5 |
| hsa-mir-184 | UGGACGGAGAACUGAUAAGGGU | 6 |
| hsa-mir-30a-5p | UGUAAACAUCCUCGACUGGAAG | 7 |
| hsa-mir-193 | AACUGGCCUACAAAGUCCCAG | 8 |
| hsa-mir-193a | AACUGGCCUACAAAGUCCCAGU | 9 |
| hsa-mir-200c | UAAUACUGCCGGGUAAUGAUGGA | 10 |
| hsa-mir-328 | CUGGCCCUCUCUGCCCUUCCGU | 11 |
| hsa-mir-330 | GCAAAGCACACGGCCUGCAGAGA | 12 |
| hsa-mir-409-5p | AGGUUACCCGAGCAACUUUGCAU | 13 |
| I-ha-min-44913 | AGGCAGUGUAUUGUUAGCUGGC | 14 |
| hsa-mir-485-3p | GUCAUACACGGCUCUCCUCUCU | 15 |
| hsa-mir-554 | GCUAGUCCUGACUCAGCCAGU | 16 |
| hsa-137 | UUAUUGCUUAAGAAUACGCGUAG | 17 |

In some cases, there could be additional family members of said miRNAs (Table 1) that are recognized in the art and which should be considered equivalents of the specific sequences listed herein. Although all sequences are shown as RNA sequences, it will be understood that, when referring to hybridizations or other assays, corresponding DNA sequences can be used as well. For example, RNA sequences may be reverse transcribed and amplified using the polymerase chain reaction (PCR) in order to facilitate detection. In those cases, it will actually be DNA and not RNA that is directly quantified. It will also be understood that the complement of the reverse transcribed DNA sequences can be analyzed instead of the sequence itself. In this context, the term “complement” refers to an oligonucleotide that has an exactly complementary sequence, i.e. for each adenine (A) there is a thymine (T), etc. Although assays may be performed for the miRNAs individually, it is generally preferable to assay several miRNAs.

The level of expression of said miRNAs (Table 1) can be determined by any suitable method including generic methods for the detection and quantification of nucleic acids especially RNA, optimized methods for the detection and quantification of small RNA species, as both mature and precursor miRNAs fall into this category as well as specially designed methods for the detection and quantification of miRNA species. Illustrative, non-limitative, examples of methods which can be used to facilitate the testing of multiple miRNAs include:
1) multiplex and/or singleplex real-time RT-PCR (reagents available from, e.g., Applied Biosystems and System Biosciences (SBI)), including quantitative real time reverse transcriptase PCR (qRT-PCR) as described in U.S. Pat. No. 5,928,907 and U.S. Pat. No. 6,015,674;
2) single-molecule detection as described by Neely, et al., Nat. Methods. 3(1):41-6 (2006) and in U.S. Pat. No. 6,355,420; U.S. Pat. No. 6,916,661; and U.S. Pat. No. 6,632,526;
3) bead-based flow cytometric methods as described by Lu, et al., Nature 435:7043 (2005) and in U.S. Pat. Nos. 6,524,793; and
4) assays using arrays of nucleic acids as described by Nelson, et al., Nat. Methods 1(2):155-61 (2004); Wu, et al., RNA 13(1):151-9 (2007) and in U.S. Pat. No. 6,057,134; U.S. Pat. No. 6,891,032; U.S. Pat. No. 7,122,303; U.S. Pat. No. 6,458,583; U.S. Pat. No. 6,465,183; U.S. Pat. No. 6,461,816; U.S. Pat. No. 6,458,583; U.S. Pat. No. 7,026,124; U.S. Pat. No. 7,052,841; U.S. Pat. No. 7,060,809; U.S. Pat. No. 6,436,640; and U.S. Pat. No. 7,060,809.

Microarrays can also be prepared in which oligonucleotides having complementary sequences (or oligonucleotides with sequences matching the miRNAs themselves) are immobilized on the surface of a solid support. Materials that can be used as supports include membranes, dishes or slides made of glass or plastic. At least 1, and, preferably, 2, 3, 4, 5, 6, 7, 8, 9, 10, 11, 12, 13, 14, 15, 16 or 17 of the miRNAs described above (Table 1) should be recognized by the immobilized oligonucleotides, with each different oligonucleotide occupying a distinct and known position on the support. Microarrays of this type may be made by using methodology well known in the art or appropriate miRNA arrays can be purchased commercially, e.g., from Ambion, Agilent or Exiqon. MiRNA can then be isolated from the sample of the subject (e.g., using Ambion's Leucolock kit (AM1923)), amplified by using, e.g., the polymerase chain reaction (PCR), and analyzed by hybridizations performed under stringent conditions. The term “stringent conditions” indicates conditions that essentially only permit hybridization to occur with the exact complementary sequence of the immobilized oligonucleotide. In general, these hybridizations are performed in buffers of about neutral pH containing 0.1-0.5 NaCl and at a temperature of between 37° C. to 50° C. It is also possible to carry out incubations under conditions of low stringency and then to use high stringency wash conditions to cause the dissociation of hybridized sequences that are not exact matches.

One way to carry out microarray assays would involve amplifying miRNA in the presence of a detectable label, e.g., a nucleotide bound to a dye or other marker and present in a PCR primer. Thus, a population of labeled cDNAs may be obtained that can be used directly in hybridizations with oligonucleotides immobilized on a microarray plate or slide. After hybridizations are completed, plates may be analyzed using an automated reader to determine the amount of label associated with each immobilized sequence, which, in turn, reflects the abundance of the hybridized sequence in the original miRNA population. Many variations of this basic procedure have been described in the art and are compatible with the present invention.

In a particular embodiment, the expression level of one or more of the following miRNA: hsa-mir-18b, hsa-mir-493, hsa-mir-599, hsa-mir-96, hsa-mir-148a, hsa-mir-184, hsa-mir-30a-5p, hsa-mir-193, hsa-mir-193a, hsa-mir-328, hsa-mir-409-5p, hsa-mir-449b, hsa-mir-485-3p, hsa-mir-200c, hsa-mir-330, hsa-mir-554, and/or hsa-mir-137 is determined by real-time quantitative RT-PCR (qRT-PCT), a modification of the polymerase chain reaction (PCR) used to rapidly measure the quantity of a product of the PCR. It is preferably done in real-time, thus it is an indirect method for quantitatively measuring starting amounts of DNA, complementary DNA or RNA. This is commonly used for the purpose of determining whether a genetic sequence is present or not, and if it is present the number of copies in the sample. Like other forms of PCR, the process is used to amplify DNA samples, using thermal cycling and a thermostable DNA polymerase. The three commonly used methods of quantitative polymerase chain reaction are through agarose gel electrophoresis, the use of SYBR Green (a double stranded DNA dye), and the fluorescent reporter probe. The latter two of these three can be analysed in real-time, constituting real-time polymerase chain reaction method.

Using SYBR Green dye gives results in real time. A DNA binding dye binds all newly synthesized double stranded (ds)DNA and an increase in fluorescence intensity is measured, thus allowing initial concentrations to be determined. However, SYBR Green will label all dsDNA including any unexpected PCR products as well as primer dimers, leading to potential complications and artefacts. The reaction is prepared as usual, with the addition of fluorescent dsDNA dye. The reaction is run, and the levels of fluorescence are monitored; the dye only fluoresces when bound to the dsDNA. With reference to a standard sample or a standard curve, the dsDNA concentration in the PCR can be determined.

The fluorescent reporter probe method is the most accurate and most reliable of the methods. It uses a sequence-specific nucleic acid based probe so as to only quantify the probe sequence and not all double stranded DNA. It is commonly carried out with DNA based probes with a fluorescent reporter and a quencher held in adjacent positions, so-called dual-labelled probes. The close proximity of the reporter to the quencher prevents its fluorescence; it is only on the breakdown of the probe that the fluorescence is detected. This process depends on the 5′ to 3′ exonuclease activity of the polymerase involved. The real-time quantitative PCR reaction is prepared with the addition of the dual-labelled probe. On denaturation of the double-stranded DNA template, the probe is able to bind to its complementary sequence in the region of interest of the template DNA (as the primers will too). When the PCR reaction mixture is heated to activate the polymerase, the polymerase starts synthesizing the complementary strand to the primed single stranded template DNA. As the polymerisation continues it reaches the probe bound to its complementary sequence, which is then hydrolysed due to the 5′-3′ exonuclease activity of the polymerase thereby separating the fluorescent reporter and the quencher molecules. This results in an increase in fluorescence, which is detected. During thermal cycling of the real-time PCR reaction, the increase in fluorescence, as released from the hydrolysed dual-labelled probe in each PCR cycle is monitored, which allows accurate determination of the final, and so initial, quantities of DNA.

Any method of PCR that can determine the expression of a nucleic acid molecule as defined herein falls within the scope of the present invention. A preferred embodiment of the present invention regards the real-time quantitative RT-PCR method, based on the use of either SYBR Green dye or a dual-labelled probe for the detection and quantification of nucleic acids according to the herein described. A more preferred embodiment of the present invention regards the methods of real-time quantitative RT-PCR for the expression profiling of miRNAs in MS.

The results obtained are compared with those obtained using reference samples. Nevertheless, it will be understood that it is not absolutely essential that an actual reference sample be run at the same time that assays are being performed on a test sample. Once reference (normal) levels of the miRNAs have been established, said levels can provide a basis for comparison without the need to rerun a new reference sample with each assay. The comparison between the test and reference samples provides a basis for a conclusion as to whether a subject has MS.

Effectively, a statistically significant deregulation in the level of expression of said miRNA (e.g., hsa-mir-18b, hsa-mir-493, hsa-mir-599, hsa-mir-96, hsa-mir-148a, hsa-mir-184, hsa-mir-193, hsa-mir-193a, hsa-mir-328, hsa-mir-409-5p, hsa-mir-449b, hsa-mir-485-3p, hsa-mir-200c, hsa-mir-330, hsa-mir-554 and/or hsa-mir-137) in the subject sample with respect to the normal level of said miRNA(s) in the reference sample is indicative that the subject is afflicted with MS.

As used herein the term “deregulation” means that the expression of the miRNA is altered and includes “upregulation” (overexpression) and “downregulation” (underexpression). A miRNA is upregulated when its expression is increased in comparison with its normal level in a reference sample. Similarly, a miRNA is downregulated when its expression is decreased in comparison with its normal level in a reference sample. In general, the greater the difference between the test sample and the reference sample, the stronger the indication for the presence of MS. Thus, the expression level of the MS diagnostic miRNA (Table 1) once determined in the test sample is compared with the expression level of said miRNAs in a reference sample; at a minimum, a difference of about 10% between the expression levels in each sample should be seen to conclude that a disease (MS) is present; higher differences, e.g., about 15%, about 20%, about 25%, about 30%, about 50%, about 60%, about 75%, about 100%, or more (e.g., about 150%), being more conclusive.

In a particular embodiment, the method of the invention comprises comparing:
a) the level of expression of a miRNA selected from the group consisting of hsa-mir-18b, hsa-mir-493, hsa-mir-599, hsa-mir-96, hsa-mir-148a, hsa-mir-184, hsa-mir-193, hsa-mir-193a, hsa-mir-328, hsa-mir-409-5p, hsa-mir-449b, hsa-mir-485-3p, hsa-mir-200c, hsa-mir-330, hsa-mir-554, hsa-mir-137 and combinations thereof, in a sample from said subject; and
b) the normal level of expression of said miRNA(s) in a reference sample,
   wherein a statistically significant increase in the level of expression of said miRNA hsa-mir-18b, hsa-mir-493, hsa-mir-96, hsa-mir-148a, hsa-mir-193, hsa-mir-193a, hsa-mir-328, hsa-mir-409-5p, hsa-mir-449b or hsa-mir-485-3p, in the subject sample with respect to the normal level of said miRNA(s) in the reference sample is indicative that the subject is afflicted with MS, or
   wherein a statistically significant decrease in the level of expression of said miRNA hsa-mir-599, hsa-mir-184, hsa-mir-200c, hsa-mir-330, hsa-mir-554 or hsa-mir-137 in the subject sample with respect to the normal level of said miRNA(s) in the reference sample is indicative that the subject is afflicted with MS.

With respect to hsa-mir-599 and hsa-mir-184, Example 1 shows that a statistically significant increase in the level of expression of said miRNAs in the subject sample with respect to the normal level of said miRNA(s) in the reference sample is indicative that the subject is afflicted with MS. However, Example 2, confirms that said miRNAs (hsa-mir-599 or hsa-mir-184) have an altered expression in MS patients vs controls although the direction of the deregulation (up- or down-regulated) is not the same as in Example 1 for said miRNAs. Whereas in Example 1 hsa-mir-599 and hsa-mir-184 are both upregulated in MS patients vs controls, in Example 2 said miRNAs hsa-mir-599 and hsa-mir-184 are both downregulated in MS patients vs controls. These differences may be explained by the increase in the number of samples (21 in Example 1 and 68 in Example 2). An increase in the number of sample allows to verify the direction of the deregulation of the miRNAs which may be a constant in the disease or may depend from other factors (e.g., genetic background of the patient, treatment, etc.).

The above-identified miRNAs (Table 1) are particularly useful in the diagnosis of MS.

In a particular embodiment, said miRNAs are one or more of hsa-mir-493, hsa-mir-193, hsa-mir-193a, hsa-mir-328, hsa-mir-409-5p, hsa-mir-449b, hsa-mir-485-3p and hsa-mir-96; said miRNAs showing a differential expression (deregulation) in subjects afflicted with MS experiencing a relapse (i.e., increased or decreased levels of some miRNAs vs controls are indicative of the presence of MS in a relapse status); most preferably, said miRNA is selected from the group consisting of hsa-mir-493, hsa-mir-193, hsa-mir-193a, hsa-mir-328, hsa-mir-409-5p, hsa-mir-449b, hsa-mir-485-3p, hsa-mir-96 and combinations thereof.

Example 1 shows that a statistically significant increase in the level of expression of miRNAs hsa-mir-18b and hsa-mir-599 in the subject sample with respect to the normal level of said miRNA(s) in the reference sample is indicative that the subject afflicted with MS is experiencing a relapse. However, Example 2, confirms that said miRNAs (hsa-mir-18b and hsa-mir-599) have both an altered expression in MS patients in relapse vs controls although the direction of the deregulation (up- or down-regulated) is not the same as in Example 1 for said miRNAs. Thus, whereas in Example 1 said miRNAs hsa-mir-18b and hsa-mir-599 are both upregulated in MS patients experiencing a relapse vs controls, in Example 2 said miRNAs hsa-mir-18b and hsa-mir-599 are both downregulated in MS patients in relapse vs controls. As mentioned above, this difference may be explained by the increase in the number of samples which allows to verify the direction of the deregulation of the miRNAs and depend from a lot of factors.

In another particular embodiment, said miRNAs are one or more of hsa-mir-96, hsa-mir-148a, hsa-mir-30a-5p, hsa-mir-193, hsa-mir-193a, hsa-mir-328, hsa-mir-409-5p, hsa-mir-449 and hsa-mir-485-3p, said miRNAs showing a differential expression in subjects afflicted with MS experiencing a remission (i.e., increased levels of said miRNAs are indicative of the presence of MS in a remission status); most preferably, said miRNA is hsa-mir-96.

Example 2 shows that a statistically significant decrease in the level of expression of miRNA hsa-mir-184 in the subject sample with respect to the normal level of said miRNA in the reference sample is indicative that the subject afflicted with MS is experiencing a remission. However, Example 1, shows that said miRNA (hsa-mir-184) has an altered expression in MS patients in remission vs controls although the direction of the deregulation (up- or down-regulated) is not the same as in Example 2 for said miRNA. Thus, whereas in Example 2 hsa-mir-184 is downregulated in MS patients experiencing a remission vs controls, in Example 1 said miRNA hsa-mir-184 is upregulated in MS patients in remission vs controls. This difference may be explained by the increase in the number of samples for the reasons previously mentioned.

Therefore, in another aspect, the invention relates to a method for assessing if a subject afflicted with Multiple Sclerosis (MS) is experiencing a relapse, which comprises comparing:
a) the level of expression of a miRNA selected from the group consisting of hsa-mir-493, hsa-mir-193, hsa-mir-193a, hsa-mir-328, hsa-mir-409-5p, hsa-mir-449b, hsa-mir-485-3p, hsa-mir-96 and combinations thereof, in a test sample from said subject; and
b) the normal level of expression of said miRNA(s) in a reference sample, wherein a statistically significant increase in the level of expression of said miRNA(s) in the subject sample with respect to the normal level of said miRNA(s) in the reference sample is indicative that the subject afflicted with MS is experiencing a relapse.

“Relapse” could be defined as the appearance of new symptoms or the aggravation of old ones, lasting at least twenty-four hours (synonymous with attack, relapse, flare-up, or worsening), usually associated with inflammation and demyelination in the brain or spinal cord. To be a true exacerbation, the attack must last at least 24 hours and be separated from the previous attack by at least 30 days. Most exacerbations last from a few days to several weeks or even months.

The particulars of the samples, both the test and the reference samples, have been previously mentioned in connection with the method of the invention. The expression levels of said miRNAs (hsa-mir-493, hsa-mir-193, hsa-mir-193a, hsa-mir-328, hsa-mir-409-5p, hsa-mir-449b, hsa-mir-485-3p, hsa-mir-96, and combinations thereof) can be determined as discussed in connection with the method of the invention as well as what should be considered as a statistically significant increase.

Further, in another aspect, the invention relates to a method for assessing if a subject afflicted with Multiple Sclerosis (MS) is experiencing a remission, which comprises comparing:
a) the level of expression of a miRNA selected from the group consisting of hsa-mir-96, hsa-mir-148a, hsa-mir-193, hsa-mir-193a, hsa-mir-328, hsa-mir-409-5p, hsa-mir-449b, hsa-mir-485-3p, and combinations thereof, in a test sample from said subject; and
b) the normal level of expression of said miRNA(s) in a reference sample, wherein a statistically significant increase in the level of expression of said miRNA(s) in the subject sample with respect to the normal level of said miRNA(s) in the reference sample is indicative that the subject afflicted with MS is experiencing a remission.

“Remission” could be defined as a lessening in the severity of symptoms or their temporary disappearance during the course of the illness. It also refers to a patient with RR-MS without clinical symptoms of a relapse.

The particulars of the samples, both the test and the reference samples, have been previously mentioned in connection with the method of the invention. The expression levels of said miRNAs (hsa-mir-96, hsa-mir-148a, hsa-mir-193, hsa-mir-193a, hsa-mir-328, hsa-mir-409-5p, hsa-mir-449b, hsa-mir-485-3p, and combinations thereof) can be determined as discussed in connection with the method of the invention as well as what should be considered as a statistically significant increase.

Further, in another aspect, the invention relates to a method for assessing if a subject afflicted with Multiple Sclerosis (MS) is experiencing a remission, which comprises comparing:
a) the level of expression of a miRNA selected from the group consisting of hsa-mir-18b, hsa-mir-493, hsa-mir-599, and combinations thereof, in a test sample from said subject; and
b) the level of expression of said miRNA(s) in a control sample, said control sample being a sample from the same subject under analysis obtained during a period in which said subject afflicted with MS under analysis is experiencing a relapse,
   wherein a statistically significant decrease in the level of expression of said miRNA(s) in the subject sample with respect to the level of said miRNA(s) in the control sample is indicative that the subject afflicted with MS is experiencing a remission.

The particulars of the test samples have been previously mentioned in connection with the method of the invention. The expression levels of said miRNAs (hsa-mir-18b, hsa-mir-493, hsa-mir-599, and combinations thereof) can be determined as discussed in connection with the method of the invention. According to this aspect of the invention, the level of expression of said miRNA(s) in a test sample from the subject under analysis is compared to the level of expression of said miRNA(s) in a control sample, said “control” sample being a sample from the same subject under analysis obtained during a period in which said subject afflicted with MS under analysis is experiencing a relapse. If a subject afflicted with MS is experiencing a relapse can be assessed as mentioned above. The comparison between the test and control samples provides a basis for a conclusion as to whether a subject afflicted with MS is experiencing a remission. A difference of about 10% between the expression level of said miRNA in the test sample with respect to the control sample should be seen to conclude that a subject afflicted with MS is experiencing a remission; higher differences, e.g. about 15%, about 20%, about 25%, about 30%, about 50%, about 60%, about 75%, or more, being more conclusive.

Further, in another aspect, the invention relates to a method for assessing if a subject afflicted with Multiple Sclerosis (MS) is experiencing a relapse, which comprises comparing:
a) the level of expression of a miRNA selected from the group consisting of hsa-mir-96, hsa-mir-148a, hsa-mir-184, hsa-mir-193, hsa-mir-18b, hsa-mir-599, hsa-mir-200c, hsa-mir-330, hsa-mir-554, hsa-mir-137 and combinations thereof, in a test sample from said subject; and
b) the level of expression of said miRNA(s) in a control sample, said control sample being a sample from the same subject under analysis obtained during a period in which said subject afflicted with MS under analysis is experiencing a remission,
   wherein a statistically significant decrease in the level of expression of said miRNA(s) in the subject sample with respect to the level of said miRNA(s) in the control sample is indicative that the subject afflicted with MS is experiencing a relapse.

The particulars of the test samples have been previously mentioned in connection with the method of the invention. The expression levels of said miRNAs (hsa-mir-96, hsa-mir-148a, hsa-mir-184, hsa-mir-193, hsa-mir-18b, hsa-mir-599, hsa-mir-200c, hsa-mir-330, hsa-mir-554, hsa-mir-137 and their combinations) can be determined as discussed in connection with the method of the invention. According to this aspect of the invention, the level of expression of said miRNA(s) in a test sample from the subject under analysis is compared to the level of expression of said miRNA(s) in a control sample, said “control” sample being a sample from the same subject under analysis obtained during a period in which said subject afflicted with MS under analysis is experiencing a remission. If a subject afflicted with MS is experiencing a remission can be assessed as mentioned above. The comparison between the test and control samples provides a basis for a conclusion as to whether a subject afflicted with MS is experiencing a relapse. A difference of about 10% between the expression level of said miRNA in the test sample with respect to the control sample should be seen to conclude that a subject afflicted with MS is experiencing a relapse, with higher differences, e.g., about 15%, about 20%, about 25%, about 30%, about 50%, about 60%, about 75%, or more, being more conclusive.

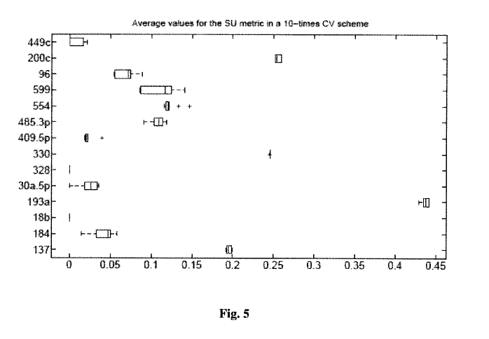

In a particular embodiment, as shown in Example 2, in the non-parametric approach, the symmetrical uncertainty (SU) analysis outputed 4 miRNAs (miR-200c, miR-330, miR-193a and miR-137) that significantly surpassed the rest, and so, it can be stated that said four miRNAs are the most relevant features in the classification problem (FIG. 4). So, in order to evaluate the potential of these miRNAs as biomarker of the disease, the data were discretized by the inventors using the equal width method, dividing the expression levels into three intervals. Afterwards, 3 datasets according to the SU results were created:
1. All the studied miRNAs: 14 miRNAs.
2. miRNAs 200c, 96, 599, 554, 485-3p, 330, 193a and 137, discarding those miRNAs with the lowest correlation level with the class (very low average SU metric value): 8 miRNAs out of 14.
3. miRNAs with the highest correlation with the class (highest SU value): miR-200c, 330, 193a and 137): 4 miRNAs out of 14.

Later several classifiers of very different nature were applied to those 3 datasets: Bayesian network classifiers, decision trees, logistic regression, distance based classifiers and neural networks. The evaluation of the classifiers was made in terms of the accuracy, sensitivity and specificity (Table 10) [Example 2].

Therefore, in a particular embodiment, as shown in Example 2, said mRNA is selected from the group consisting of miRNAs miR-200c, miR-330, miR-193a, miR-137 and combinations thereof.

Additionally, the teachings of the invention can be used to design a therapy to the subject afflicted with MS. Thus, in a further aspect, the invention relates to a method of designing a therapy for a subject afflicted with Multiple Sclerosis (MS), which comprises:
determining if a subject afflicted with MS is experiencing a relapse, and selecting a drug suitable for treatment of MS in a relapse status; or, alternatively,
determining if a subject afflicted with MS is experiencing a remission, and selecting a drug suitable for treatment of MS in a remission status.

If a subject afflicted with MS is experiencing a relapse can be determined according to the above mentioned method. Illustrative, non-limitative examples of drugs suitable for treatment of MS in a relapse status include corticosteroids, etc. In fact, during symptomatic attacks, administration of high doses of intravenous corticosteroids, such as methylprednisolone, is the routine therapy for acute relapses.

If a subject afflicted with MS is experiencing a remission can be determined according to the above mentioned method. Illustrative, non-limitative examples of drugs suitable for treatment of MS include interferon beta-1a (e.g., marketed under trade names Avonex, CinnoVex, ReciGen or Rebif) and one of interferon beta-1b (marketed in the US under trade name Betaseron, and in Europe and Japan as Betaferon), glatiramer acetate (Copaxone), mitoxantrone, and natalizumab (marketed under trade name Tysabri).

The following Examples have been included to illustrate some modes of the presently-disclosed subject matter. In light of the present disclosure and the general level of skill in the art, those of skill will appreciate that the following Examples are intended to be exemplary only and that numerous changes, modifications, and alterations can be employed without departing from the scope of the presently-disclosed subject matter.

### Example 1

### Differential miRNA Expression in PBMC from MS Patients

Inventors have studied the expression pattern of 364 microRNA in peripheral blood mononuclear cells (PBMC) obtained from multiple sclerosis patients in relapse status, in remitting status and healthy controls with the aim of understanding the regulatory mechanisms of said stages. Expression values were obtained by means of qPCR. The most relevant miRNA were validated in art independent set of samples. In order to determine the effect of the miRNA pattern, the expression of some predicted target genes were studied by qPCR. Gene interaction networks were constructed in order to obtain a system biology and multivariate view of the experimental data. The data analysis and later validation reveal that some miRNAs, e.g., hsa-mir-18b, hsa-mir-493 and hsa-mir-599, specially hsa-mir-18b and hsa-mir-599, may be relevant at the time of relapse and that other miRNA (hsa-mir-96, hsa-mir-148a, hsa-mir-184, has-mir-30a-5p and hsa-mir-193, specially hsa-mir-96, may be involved in the remitting phenomena. This study highlights the importance of the miRNA expression in the molecular mechanisms implicated in the disease.

### 1. Materials and Methods

### Recruitment of Individuals

All patients were recruited in the Neurology Department of Hospital Donostia, located in the region of Gipuzkoa (Basque Country, Spain). The study was approved by the local institutional review board and all the samples were obtained with the written informed consent of the subjects. The patients were diagnosed as having MS according to the McDonald Criteria [McDonald, W. et al. Recommended diagnostic criteria for multiple sclerosis: guidelines from the International Panel of the diagnosis of multiple sclerosis. Ann. Neural. 50, 121-127 (2001); Poser, C. M. Revisions to the 2001 McDonald diagnostic criteria. Ann. Neural. 59, 727-728 (2006)].

In a first group (Group A), 21 blood samples were obtained: 9 from patients in remission, 4 from patients during a relapse before the administration of steroids and 8 from healthy volunteers. Total RNA, including miRNA, was extracted from these samples to carry out the study.

Samples were collected from two other non-related groups to validate independently some of the results obtained:
Group B: mRNA was obtained from 42 samples; 14 in remitting status, 13 in relapse status and 15 controls.
Group C: miRNA was extracted from 14 samples; 4 in relapse, 3 in remitting status and 7 healthy controls.

Demographic data of the individuals are shown in Table 2.

Blood extraction was always performed in the early morning and RNA extraction was carried out no more than 2 hours after the blood was collected.

TABLE 2
Demographic data of individuals

| sample | Status | Stage | Sex | Age | | Tev | age at onset | EDSS | Relapse Symptoms | te |
|---|---|---|---|---|---|---|---|---|---|---|
| 1 | EM | Relapse | Male | 35 | | 1 | 34 | 3 | Sensitive | 2 |
| 2 | EM | Relapse | Male | 53 | | 10 | 43 | 4.5 | Paraparesy and muscle weakness | 1 |
| | | | | | | | | | in Right leg and inestability | |
| 3 | EM | Relapse | Male | 38 | | 11 | 27 | 3.5 | brote medular | 1 |
| 4 | EM | Relapse | Female | 46 | 43 ± 8.1 | 19 | 27 | 5 | Spasticity in legs and dificulty | 2 |
| | | | | | | | | | in moving | |
| 5 | CON | | Male | 33 | | | | | | |
| 6 | CON | | Female | 34 | | | | | | |
| 7 | CON | | Female | 28 | | | | | | |
| 8 | CON | | Female | 35 | | | | | | |
| 9 | CON | | Female | 32 | | | | | | |
| 10 | CON | | Female | 26 | | | | | | |
| 11 | CON | | Female | 28 | | | | | | |
| 12 | CON | | Male | 33 | 31.13 ± 3.3 | | | | | |
| 13 | EM | Remitting | Female | 45 | | 22 | 23 | 2.5 | | |
| 14 | EM | Remitting | Female | 37 | | 7 | 30 | 1.5 | | |
| 15 | EM | Remitting | Female | 54 | | 11 | 43 | 2 | | |
| 16 | EM | Remitting | Female | 45 | | 12 | 33 | 6 | | |
| 17 | EM | Remitting | Female | 69 | | 22 | 47 | 2 | | |
| 18 | EM | Remitting | Female | 41 | | 20 | 21 | 3.5 | | |
| 19 | EM | Remitting | Female | 38 | | 2 | 36 | 2 | | |
| 20 | EM | Remitting | Male | 33 | | 11 | 21 | 6 | | |
| 21 | EM | Remitting | Male | 45 | 45.2 ± 11 | 5 | 40 | 2 | | |

Clinical description of the patients.
Tev: Time of evolution (years).
EDSS: Expanded Disability Status Score.
te: Time from the relapse onset and the blood extraction (in days)

RNA Extraction, Reverse Transcription (RT) and Quantitative PCR (qPCR)

Total RNA was extracted from blood using the Ambion Leucolock kit (AM1923) working with the alternative protocol so as to keep the small RNA fraction. The RNA obtained was quantified in triplicate using a NanoDrop spectrophotometer (NanoDrop Technologies, USA). A common bias in the interpretation of the miRNA profiles from whole blood may be introduced by the high concentration of miRNA from erythrocytes (Chen, S. Y., Wang, Y., Telen, M. J., & Chi, J. T. The genomic analysis of erythrocyte microRNA expression in sickle cell diseases. PLoS. ONE. 3, e2360 (2008)]. In this study, such a bias was avoided by isolating PBMC in a filter prior to RNA purification.

In the case of group B, the samples came from RNA samples that are being collected systematically by the inventors' group. They were extracted using a Versagene™ Kit (Gentra, Minneapolis, USA). This kind of extraction method entails the loss of the small molecules of RNA, i.e. miRNA.

The cDNA was synthesized from total RNA using a Multiplex RT for Taqman array kit (Applied Biosystems, Foster City, Calif.). Briefly, this kit consists of 8 pre-defined RT primer pools containing up to 48 RT primers each. Each of these 8 pools contains the same endogenous controls (RNU48). This technology has been developed to detect only full length mature miRNA but not their precursors or their partially-degraded products.

A qPCR was performed by using the Taqman® Low Density Array (TLDA) Human MicroRNA Panel v1.0 from Applied Biosystems. This TLDA included 365 miRNA assays plus an endogenous control. The qPCR was performed using an Applied Biosystems 7900 Sequence Detection System. Ct values were determined using the automatic threshold in RQ manager v1.1 analysis software.

Two normalization steps were used: the first normalization consisted in loading the same quantity of template RNA and the second in normalizing the data against an endogenous gene. This endogenous control (RNU48) was chosen for this study as the least variable of all endogenous genes included in the TLDA assays. Consequently, data was normalized to RNU48, using the values of each of the 8 pools, i.e. each gene pool was normalized against the endogenous gene that was convened to cDNA in the same pool, to avoid introducing bias in the results.

Relative quantification of miRNA expression was calculated with the 2^{-ddCT} method (Applied Biosystems User Bulletin No 2 (P/N 4303859)). Quality of the data and quantification was computed using Real-Time Statminer© software (www.integromics.com). This software performs a classical t-test between the groups (relapse, remitting and control) and corrects them using the Benjamini-Hochberg algorithm [Benjamini, Y. & Hochberg, Y. Controlling the false discovery rate: A practical and powerful approach to multiple testing, J. Roy. Statist. Soc. Ser B 57, 289-300 (1995)] with the False Discovery Rate (FDR) set at a value of 5%.

### Statistical Data Analysis

A non-parametric analysis that complements the classical t-test analysis was performed trying to reveal alternative results over the low number of available samples. The expression patterns of the three groups was compared by pairs: relapse vs remitting, relapse vs control and remitting vs control. To accomplish this task, a non-parametric ranking method called Symmetrical Uncertainty (SU) sorts all the miRNA according to their statistical relevance over each of the three comparisons using the following coefficient:

$$SU ( Y , C ) = \frac{2 ( H ( Y ) - H ( Y \vert C ) )}{H ( Y ) + H ( C )}$$

wherein

Y is the predictive variable (in this case, each miRNA),

C is the class label to be predicted (depending on the comparison carried out, it takes two of the following three values: remitting, relapse and control),

H(Y) is the entropy of Y and H(Y|C) is the conditional entropy of Y given C [Cover T M & Thomas J A, Elements of Information Theory (Wiley Interscience, 2006)].

The SU ranking is based on the mutual information between each miRNA expression level and the phenotype label. Being a univariate coefficient, it measures the uncertainty reduction of the class variable C when the expression value of an miRNA (denoted as Y in the above formulation) is known. As the SU metric only deals with discrete/categorical variables, the DCT expression of each miRNA was firstly discretized into three intervals by means of an equal width discretization.

In order to get a system biology and multivariate view of the experimental data, the inventors undertook the construction of co-expression networks to investigate the possible regulations within two out of our three comparisons (relapse vs remitting and remitting vs control). For this purpose, the inventors borrowed a technique for building gene interaction networks [Armananzas, R., Inza, I., & Larrañaga, P. Detecting reliable gene interactions by a hierarchy of Bayesian network classifiers. Computer methods and programs in biomedicine 91, 110-121 (2008)] and applied it to their DCT expression data. This algorithm makes use of three main components to find reliable dependences from data: a bootstrap re-sampling algorithm, a Bayesian network classifier and a dimensionality reduction technique. The algorithm's construction scheme is focused on finding highly reliable dependences from raw data. The bootstrap step re-samples the original data B times, obtaining B similar datasets. For each sampled dataset a dimensionality reduction step is made using the correlation-based filter selection approach (CFS) [Hall M A & Smith L A Feature subset selection: a correlation based filter approach. Proceedings of the Fourth International Conference on Neural Information Processing and Intelligent Information Systems 855-858 (1997)]. The CFS returns sets of relevant features that show a high degree of correlation with the class label while the redundancy degree among them is kept as low as possible. Each sampled dataset is projected to contain only the selected features and afterwards a k-dependence Bayesian network classifier is induced from that data [Sahami, M. Learning limited dependence Bayesian classifiers, in Proceedings of the Second International Conference on Knowledge Discovery and Data Mining 335-338, 1996]. All the probabilistic dependences found by the B final classifier are stored.

The algorithm's output is a hierarchy of probabilistic dependences found during the whole process. When a cut-off threshold T is set, it is possible to retrieve a graphical structure in which only those probabilistic conditional dependences that have been configured at least T times are displayed. Each arc in the final structure is associated with a robustness value which reflects the number of times the arc is configured in the different bootstrap re-samplings.

A total of 10,000 re-samplings was performed with their correspondent CFS and k-DB data mining techniques. The value of k in k-DB was set to four, keeping to the value suggested in the original work.

Sequence of the miRNA Genes

The selected miRNA genes were amplified by PCR (primers sequences available on request) and the PCR product was sequenced in an ABI3130 automatic sequencer (Applied Biosystems) using Bigdye v3.1. The used primers were designed based on the mirbase [Griffiths-Jones, S. The microRNA Registry. Nucleic Acids Res. 32, D109-D111 (2004); Griffiths-Jones, S. miRBase: the microRNA sequence database. Methods Mol. Biol. 342, 129-138 (2006); Griffiths-Jones, S., Saini, H. K., van, D. S., & Enright, A. J. miRBase: tools for microRNA genomics. Nucleic Acids Res. 36, D154-D158 (2008)] sequence information and using the Generunner software (www.generunner.com). Group A samples (n=21) were analyzed as well as 40 healthy controls.

### Validation of the Target Genes

An independent set of 42 samples (group B) was studied. The expression was analyzed by qPCR using SYBRgreen as fluorescent and pre-designed primers from geneglobe (www.geneglobe.com). The assay codes can be found in Table 3. The data were analyzed using the same software and the same methodology described above, using as the endogenous gene GAPDH. The expression of four selected genes was studied. Table 3 presents the miRNAs that joint to these genes and the group in which it is expected to be down-regulated.

TABLE 3
Target genes studied with their gene ID, the miRNA that
binds to the gene, the group in which these genes are expected
to be down-regulated and the Geneglobe Assay code

| | | | Expected to | |
|---|---|---|---|---|
| | Gene | | be down | |
| | ID | miRNA | regulated in: | Assay code |
| ARHGEF12 | 23365 | hsa-mir-96/ | Remitting group | QT00006762 |
| | | hsa-mir-148/ | | |
| | | hsa-mir-193 | | |
| CELSR2 | 1952 | hsa-mir-96 | Remitting group | QT00010948 |
| TAOK3 | 51347 | hsa-mir-599 | Relapse group | QT00059843 |
| GAB1 | 2549 | hsa-mir-18b | Relapse group | QT00014154 |

Individual Validation of the miRNA Expression

The validation of the expression of the selected miRNA genes was performed in an independent set of 14 samples (Group C). The qPCR was performed in a 7900 sequence detection system using pre-designed Taqman probes (Applied Biosystems).

### 2. Results

qPCR was used to study the expression of 364 miRNAs in samples from 4 MS patients during a relapse and from 9 patients during remission. Also 8 healthy controls were analyzed.

On average, 45% of the miRNA analyzed were expressed in any given sample. Although several miRNA reached nominal significance in the t-test, only three remained significant after correction for multiple testing (with an FDR threshold of 5%). The transcript hsa-mir-18b showed increasing expression in the relapsing group when comparing the relapse group vs the control group (RQ: 52.1). The transcripts hsa-mir-493 and hsa-mir-599 showed expression in the relapsing group whereas they were not expressed in controls. These two miRNAs are also expressed in the remitting group but did not reach any statistical significance in the comparisons.

In order to complement the information of the classical statistical analysis, the symmetrical uncertainty (SU) correlation degree of each miRNA with respect to the class phenotype was calculated, providing a ranked list of all miRNAs. The top ten miRNA emerging from these rankings are shown in Table 4. The rankings have been made for three different comparisons; relapse versus remitting, remitting versus controls and relapse versus controls. Highlighted are the significant miRNAs found in the previous analysis.

TABLE 4
Top 10 genes from the SU analysis for each of the three comparisons

| | Relevance as Symmetrical Uncertainty | | | | | |
|---|---|---|---|---|---|---|
| | Relap vs. Rem | | Rem vs. CON | | Relap vs. CON | |
| | gene | SU | gene | SU | gene | SU |
| 1 | hsa-miR-542-5p | 0.5277 | hsa-miR-96 | 0.4832 | hsa-miR-599 | 0.8651 |
| 2 | hsa-miR-376a | 0.4921 | hsa-miR-30a-5p | 0.2989 | hsa-miR-18b | 0.6416 |
| 3 | hsa-miR-18b | 0.4048 | hsa-miR-30e-5p | 0.2959 | hsa-miR-423 | 0.6367 |
| 4 | hsa-miR-34c | 0.4039 | hsa-miR-599 | 0.2959 | hsa-miR-125b | 0.5738 |
| 5 | hsa-miR-489 | 0.4039 | hsa-miR-193a | 0.2959 | hsa-miR-383 | 0.5392 |
| 6 | hsa-miR-554 | 0.4039 | hsa-miR-337 | 0.2959 | hsa-miR-509 | 0.5392 |
| 7 | hsa-miR-600 | 0.4039 | hsa-miR-449b | 0.2591 | hsa-miR-30e-5p | 0.5392 |
| 8 | hsa-miR-652 | 0.4039 | hsa-miR-184 | 0.2477 | hsa-miR-487b | 0.5167 |
| 9 | hsa-miR-214 | 0.3863 | hsa-miR-328 | 0.2283 | hsa-miR-222 | 0.4970 |
| 10 | hsa-miR-328 | 0.3729 | hsa-miR-146b | 0.2238 | hsa-miR-127 | 0.4965 |

[Highlighted miRNAs (has-mir-18b and has-mir-599) were also significant in the corrected t-test]

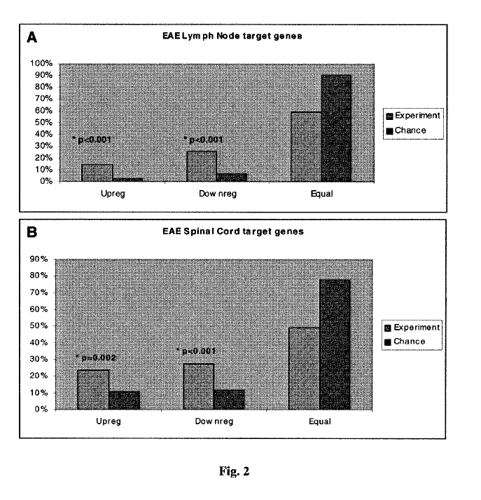

A system biology analysis was performed to obtain information about the relationships between the different miRNA. Interaction networks were built in two groups:
relapse versus remitting, in order to obtain information about the relapse phenomena in the patients; and
remitting versus controls, in order to obtain information about the MS (FIG. 2 A-B).

From these analyses of the expression data, 8 miRNAs ere selected in which further analysis were performed:
three miRNAs, hsa-mir-18b, hsa-mir-493 and hsa-mir-599, were chosen because they reached the significance level in the corrected t-test used to compare relapse status with control samples; and
five miRNAs, hsa-mir-96, hsa-mir-148a, hsa-mir-184, hsa-mir-30a-5p, and hsa-mir-193, were selected, coming from the network analysis that differentiates between remitting and control groups. From this network, inventors selected the two miRNAs with more interactions (hsa-mir-96 and has-mir-30a-5p), the two with arcs showing the highest robustness values (hsa-mir-184 with 1557 and hsa-mir-193a with 1358) and the other parent of hsa-mir-96, the hsa-mir-184.
   Validation of the miRNA Expression

To validate these results, the following three miRNA: hsa-mir-18b, hsa-mir-96 and hsa-mir-599, were studied in an independent set of samples (group C).

The has-mir-18b and has-mir-599 were up-regulated four and five times more in the relapse group than in the controls. For has-mir-96 no differences in the expression between the groups were obtained.

Sequencing of the miRNA Genes

The gene sequence of 3 (hsa-mir-96, hsa-mir-493 and hsa-mir-18b) of the 7 selected mature miRNAs was amplified and sequenced in the Group A samples and in a control group (n=40). Said three miRNAs were selected as representative of the comparisons: hsa-mir-96 in the remitting versus control study and the other two in the relapse versus remitting comparison. No polymorphisms were associated with any of the groups.

miRNA Targets

In order to provide a biological interpretation of our findings, the predicted targets of each relevant miRNA was searched in three different databases; mirbase targets v5, Targetsan v4.2 [Lewis, B. P., Shih, I. H., Jones-Rhoades, M. W., Bartel, D. P., & Burge, C. B. Prediction of mammalian microRNA targets. Cell 115, 787-798 (2003); Lewis, B. P., Burge, C. B., & Bartel, D. P. Conserved seed pairing, often flanked by adenosines, indicates that thousands of human genes are microRNA targets. Cell 120, 15-20 (2005); Grimson, A. et al. MicroRNA targeting specificity in mammals: determinants beyond seed pairing. Mol. Cell 27, 91-105 (2007)] and Pictar [Krek, A. et al. Combinatorial microRNA target predictions. Nat. Genet. 37, 495-500 (2005)].

Table 5 lists the number of predicted targets for these miRNAs according to each database (two searches with different confidence thresholds were performed in mirbase).

TABLE 5
Predicted targets for each miRNA in three databases

| | mirbase | | | | | |
|---|---|---|---|---|---|---|
| | p < 0.05 | p < 0.005 | Pictar | Targetscan | Common | |
| 18 | 775 | 327 | 151 | 149 | 14 | Relapse |
| 599 | 783 | 185 | X | 173 | 11 | vs |
| 493 | 866 | 244 | X | 496 | 3 | Control |
| 96 | 909 | 361 | 698 | 592 | 57 | Non |
| 184 | 819 | 289 | 22 | 17 | 3 | Relapse |
| 148A | 918 | 353 | 429 | 434 | 46 | vs |
| 193 | 819 | 362 | 134 | 208 | 14 | Control |

[The column labeled as “common” represents the common predicted targets in the three databases]

Theoretically, these miRNAs should inhibit the expression of a certain number of target genes. The databases offer predicted information about the targets, but there are few experimental results to support it. In our analysis we took a conservative approach, taking as target genes only the common results from the three different prediction algorithms (in the mirbase case we selected the p<0.005 column).

To validate these results, the expression in blood of four of these targets in an independent sample set (GroupB) was checked, obtaining no statistical differences in the expression pattern.

Since miRNAs are highly conserved across species [Weber, M. J. New human and mouse microRNA genes found by homology search. FEBS J. 272, 59-73 (2005); Ibañez-Ventoso, C., Vora, M., & Driscoll, M. Sequence relationships among C. elegans, D. melanogaster and human microRNAs highlight the extensive conservation of microRNAs in biology. PLoS. ONE. 3, e2818 (2008)], the murine EAE model was used to validate our findings. To this end, a large multi-tissue, longitudinal gene expression profiling dataset in mouse EAE Lymph Node [Otaegui, D. et al. Increased transcriptional activity of milk-related genes following the active phase of experimental autoimmune encephalomyelitis and multiple sclerosis. J. Immunol. 179, 4074-4082 (2007)] and spinal cord [Baranzini, S. E., Bernard, C. C., & Oksenberg, J. R. Modular transcriptional activity characterizes the initiation and progression of autoimmune encephalomyelitis. J. Immunol. 174, 7412-7422 (2005)] was mined, focusing on targets of the same miRNAs the inventors identified in their cohort of MS patients. In order to check whether said target selected genes were really related with the disease, a group of 11 miRNAs was randomly selected from those that were not differentially expressed in the first analysis.

The expression of said target genes was checked for each miRNA-gene in the disease moment (at the peak of the disease and after it) between the control group and the EAE group. The target genes were classified in four groups: not found in the dataset, up-regulated, down-regulated and equally-expressed.

The results of the found genes are summarized as percentages in FIG. 2. A chi-square analysis has been performed between the groups. The figure shows the results of the analysis for the selected genes (clear) and for the randomly selected group (dark). The analyzed target-genes were differentially distributed (p<0.001) between experimental and random group in the up-regulated and down-regulated class.

A pathway analysis was conducted in Panther [Mi, H., Guo, N., Kejariwal, A., & Thomas, P. PANTHER version 6: protein sequence and function evolution data with expanded representation of biological pathways. Nucleic Acids Res. 37, D247-D252 (2007)] database for all miRNA (experimental and random). Out of the eight miRNAs targets, only targets of hsa-mir-96 appeared significantly enriched in 8 pathways (Table 6).

TABLE 6
Pathway analysis of the hsa-mir-96 targets

| | | | ex- | | |
|---|---|---|---|---|---|
| Pathway_hsa-miR-96 targets | NCBI | 96 | pected | ratio | P-value |
| Muscarinic acetylcholine | 62 | 5 | 0.14 | 35.7 | 5.39E−05 |
| receptor 1 and 3 signaling | | | | | |
| pathway | | | | | |
| Alpha adrenergic receptor | 29 | 3 | 0.06 | 50.0 | 6.88E−03 |
| signaling pathway | | | | | |
| Unclassified | 22436 | 39 | 50.29 | 0.8 | 1.01E−02 |
| Endothelin signaling | 98 | 4 | 0.22 | 18.2 | 1.23E−02 |
| pathway | | | | | |
| Interleukin signaling | 194 | 5 | 0.43 | 11.6 | 1.29E−02 |
| pathway | | | | | |
| Wnt signaling pathway | 348 | 6 | 0.78 | 7.7 | 2.18E−02 |
| Histamine H1 receptor | 43 | 3 | 0.1 | 30.0 | 2.19E−02 |
| mediated signaling pathway | | | | | |
| Metabotropic glutamate | 44 | 3 | 0.1 | 30.0 | 2.35E−02 |
| receptor group I pathway | | | | | |
| Angiotensin II-stimulated | 53 | 3 | 0.12 | 25.0 | 4.04E−02 |
| signaling through G proteins | | | | | |
| and beta-arrestin | | | | | |

### 3. Discussion

From these data, three miRNA (hsa-mir-18b, hsa-mir-493 and hsa-mir-599) were identified showing differential expression in MS patients experiencing a relapse when compared to controls. Classic parametric tests did not detect differentially expressed miRNA between samples from patients in remission vs controls. However, a network-based approach identified 5 miRNAs (hsa-mir-96, hsa-mir-148a, hsa-mir-184, hsa-mir-30a-5p, and hsa-mir-193) that distinguished samples from these two phases of the disease.

Inventors hypothesized that if a given miRNA was over-expressed in a particular group of samples, the targets of this miRNA should be down-regulated. Said hypothesis was checked in an EAE model. Interestingly, in this model the target genes of all 8 differentially expressed miRNAs appeared significantly down-regulated more times in the targets selected by the inventors' experiment than in a random target list.

However, the same effect could be seen in the up-regulated genes. These could be a retroactive regulation of the mRNA that are regulated in a translational repression form.

A biological interpretation of miRNA function in MS is complicated by the fact that most of the miRNA targets are predicted from bioinformatics analysis and are not yet validated in biological studies. To enhance the confidence, inventors only worked with consensus targets from the three public miRNA databases.

The discussion has been structured around the groups analyzed, i.e. patients in relapse status, patients in remitting status and control samples.

### Patients in Relapse Status

The results from the t-test gave inventors three differentially expressed genes between relapse and control samples. It could be expected the same differences between relapse and remitting groups. When that was checked, hsa-mir-18b and hsa-mir-599, but not hsa-mir-493, were up-regulated in relapse, this being significant in the uncorrected p-values but not in the FDR corrected values.

In the relapse versus remitting network, two of these genes, hsa-mir-18b and hsa-mir-599 appeared correlated with a direct probabilistic relationship. Moreover, in the SU analysis, hsa-mir-18b appears in the third position in the relapse-remitting ranking. The expressions of these two genes have been validated in an independent set of samples. Taken all together, these results highlight the importance of hsa-mir-18b and hsa-mir-599 in the mechanisms of the relapse. Their role remains unclear, but should be related with the regulation of the proposed target genes.

In the analyses of the pathways in which the target genes of these two miRNA may be implicated neither the target genes of each miRNA individually nor the target genes in common between both miRNA give significant results.

The study of the target genes showed no clear inhibition, as might be expected, maybe because regulation of the miRNA is occurring at the translational level rather than at the expression level.

These results support the idea that the expression of the miRNA could be useful as a biomarker of the relapse status.

### Patients in Remitting Status

The proposed network obtained in the comparison between remitting status and control samples indicate four micro RNAs likely to be implicated in the relapse (hsa-mir-96, hsa-mir-148a, hsa-mir-184, hsa-mir-30a-5p and hsa-mir-193). The results strongly suggest that hsa-mir-96 is an important candidate for further studies. This gene is also first in the SU ranking when remitting and control groups are compared. Although the classic qPCR analysis of the expression of this gene gave no significant differences, in the data it could be appreciated that this gene is more expressed in remitting samples than in controls, and less in relapse samples than in remitting, so it seems to be characteristic of the remitting phase of the disease. In the validation with an independent set the results are the same: no differences between the groups but a similar trend in the data

The Gene Ontology approach to the target-genes of hsa-mir-96 gave a list of 8 pathways that reached the significant level. As could be expected, within this list inventors found a classic immunologic associated pathway such as Interleukin signaling pathways. Two pathways, the Metabotropic glutamate receptor group I pathway and the Muscarinic acetylcholine receptor 1 and 3 signaling pathway, related with glutamate, are also present. Glutamate has been widely related with pathological mechanisms of the MS such as exocitotoxicity [Vallejo-Illarramendi, A., Domercq, M., Perez-Cerda, F., Ravid, R., & Matute, C. Increased expression and function of glutamate transporters in multiple sclerosis. Neurobiol. Dis. 21, 154-164 (2006); Matute, C. Interaction between glutamate signalling and immune attack in damaging oligodendrocytes. Neuron Glia Biol. 3, 281-285 (2007)]. These mechanisms are related with the CNS but could be being expressed in blood by the activated T-cells.

The Wnt signaling pathway is also present in the found pathways. The Wnt gene has been proposed as an important player in the development of effector T-cells and in the activation of the regulatory T-cell [Staal, F. J., Luis, T. C., & Tiemessen, M. M. WNT signalling in the immune system: WNT is spreading its wings. Nat. Rev. Immunol. 8, 581-593 (2008)]. All these pathways may be potential subjects for more in-depth studies, but at this point, their immunological role makes our data more reliable.

MS and miRNA

A relationship between miRNA expression and MS could be expected since some of the functions attributed to the miRNA include stress response, immunomodulation [de Yebenes, V. et al. miR-181b negatively regulates activation-induced cytidine deaminase in B cells. J. Exp. Med. (2008); Baltimore, D., Boldin, M. P., O'Connell, R. M., Rao, D. S., & Taganov, K. D. MicroRNAs: new regulators of immune cell development and function. Nat. Immunol. 9, 839-845 (2008)] and neuroprotection [Nelson, P. T., Wang, W. X., & Rajeev, B. W. MicroRNAs (miRNAs) in neurodegenerative diseases. Brain Pathol. 18, 130-138 (2008). Moreover, bioinformatics-based predictions propose that 30% of the human genes are regulated by miRNAs [Ross, J. S., Carlson, J. A., & Brock, G. miRNA: the new gene silencer. Am. J. Clin. Pathol. 128, 830-836 (2007)]. Therefore, the inventors hypothesized that a sizeable proportion of the mRNA differentially expressed between samples from patients during a relapse and during remission ought to be regulated by miRNAs.

These results support the role of miRNA expression patterns in MS. The reliability of the data is supported by the different statistical approaches, by the validation in an independent cohort of samples and by the congruent results, both in the gene ontology analysis and in the animal model analysis.

Although this should be validated in a larger cohort of samples, at least one miRNA is outstanding as a potential biomarker in MS and at least two more show potential to be good targets for future biomarker studies to characterize the relapse status.

### Example 2

### miRNA as Biomarkers of Multiple Sclerosis

In a previous study performed by the inventors' group the expression of 365 miRNAs had been analyzed in a group of patients both in relapse and remission moments and a group of controls. In that work 7 miRNAs were identified as potential markers of relapse and/or disease [Otaegui, D. et al. (2009). Differential micro RNA expression in PBMC from multiple sclerosis patients. PLoS. ONE. 4, e63091. The aim of the present study is to characterize the expression of 14 miRNAs selected from the previous work in a wider group of patients and controls to find the best candidates as biomarkers of multiple sclerosis.

### 1. Materials and Methods

### Patient Recruitment

Patients included in this study have been diagnosed with relapsing-remitting multiple sclerosis (RRMS) according to McDonald criteria revised by Polman [Polman, C. H. et al. (2005). Diagnostic criteria for multiple sclerosis: 2005 revisions to the “McDonald Criteria”. Ann. Neurol. 58, 840-846]. Peripheral blood of patients and healthy controls was collected in the Neurology Department of Hospital Donostia (San Sebastian, Spain) after obtaining the informed consent. For this study, samples from 15 patients in relapse, 53 patients in remission and 36 healthy controls have been recruited. All the procedures have been approved by the hospital's ethic committee.

TABLE 7
Characteristics of patients and controls included in this study

| | Patients | | | |
|---|---|---|---|---|
| | Relapse | Remission | All | Controls |
| Sample | 15 | 53 | 68 | 36 |
| size | | | | |
| Age | 37.1 ± 9.6 | 40.7 ± 10.0 | 40.3 ± 10.4 | 43.9 ± 13.0 |
| Gender | | | | |
| Men | 6 (40%) | 17 (32%) | 23 (34%) | 18 (50%) |
| Women | 9 (60%) | 36 (68%) | 45 (66%) | 18 (50%) |
| EDSS | 2.5 (0-5) | 3.25 (0-6.5) | 3.25 (0-6.5) | — |
| (mean) | | | | |

miRNA Extraction Reverse Transcription (RT) and qPCR

After obtaining peripheral blood from patients and controls inventors isolated miRNAs from leukocytes using LeucoLOCK™ Total RNA Isolation System (AM1923, AM1933, Ambion) with the alternative protocol to capture small RNAs, following the manufacturer's instructions. Briefly, this protocol consists in capturing and stabilizing the leukocyte population of the blood in a filter so as inventors get rid of the huge amount of globin mRNAs present in reticulocytes. Then, the captured cells are lysed in the filter, and nuclease-free water and 100% ethanol are added depending on the volume recovered. After several wash and centrifugation steps the total RNA (including miRNAs) of leucocytes present in blood was obtained. The obtained RNA was quantified using a NanoDrop spectrophotometer (NanoDrop Technologies, USA).

cDNA was synthesized from total RNA using miRNA reverse transcription kit (Applied Biosystems, Foster City, Calif.) following the manufacturer's instructions. Using this protocol, in each reaction a unique miRNA cDNA is synthesized because RT primers used are specific for each miRNA.

After the cDNA synthesis a quantitative PCR (qPCR) was performed using TaqMan probes. For this work, inventors used predispensed 384-well plates where TaqMan probes for selected 14 miRNA and 2 endogenous controls were already dispensed in order to reduce pipetting errors. qPCR was performed with 6 ng of cDNA input in a HT7900 thermal cycler of Applied Biosystems.

Selection of Studied miRNA

In this work inventors selected 14 miRNAs for expression analysis based on the data obtained on a previous study performed by the inventors' group [Otaegui, D. et al. (2009). Differential micro RNA expression in PBMC from multiple sclerosis patients. PLoS. ONE. 4, e6309]. The 14 miRNAs were selected using a non-parametric approach called Correlation based Feature subset Selection (CFS). This algorithm searches a group of relevant and non-redundant features of the desired phenomena. Given a subset of features, the relevancy of the set is obtained as the mean correlation of the selected features with the class (in this case, MS, remission, relapse or control), while the redundancy is measured as the mean correlation of the features between them. These two aspects are combined into a single metric value which is proportional to the relevancy and inversely proportional to the redundancy. The algorithm looks for a subset of miRNAs that maximize this metric, resulting in a set of genes that are strongly correlated with the phenotype but non-redundant among them. Apart from these 14 selected miRNAs inventors also chose two endogenous controls based on the literature (Table 8).

TABLE 8
14 miRNA selected for expression analysis and the 2 endogenous controls
analyzed. Information about the chromosome where the gene is located
and if the miRNA is in a cluster is also displayed

| Studied | | |
|---|---|---|
| miRNA | Chrom | Clustered with |
| hsa-miR-137 | 1 | None |
| hsa-miR-554 | 1 | None |
| hsa-miR-449b | 5 | hsa-miR-449^{a} |
| hsa-miR-30a-5p | 6 | None |
| hsa-miR-96 | 7 | hsa-miR-182 y 183 |
| hsa-miR-599 | 8 | hsa-miR-875 |
| hsa-miR-200c | 12 | hsa-miR-141 |
| hsa-miR-409-5p | 14 | hsa-mir-485, -453, -154, -496, -377, |
| | | -541, -412, -369, -410 y -656 |
| hsa-miR-485-3p | 14 | hsa-mir-381, -487b, -539, -889, -544, |
| | | -655, -487a, -382, -134, -668, -453, |
| | | -154, -496, -377, -541, -409 |
| hsa-miR-184 | 15 | None |
| hsa-miR-328 | 16 | None |
| hsa-miR-193a | 17 | None |
| hsa-miR-330 | 19 | None |
| hsa-miR-18b | X | hsa-miR-106a, 20b 19b-2, 92a-2 y 363 |
| RNU44 | 1 | Endogenous control |
| RNU48 | 6 | Endogenous control |

### Data Analysis

For the relative quantification of miRNA expression inventors used the 2^{-DDCT} method (Applied Biosystems User Bulletin no 2 (P/N 4303859)). Ct values were calculated with the Enterprise platform (Applied Biosystems) and quality control of samples and data analysis were performed in Real-time StatMiner v3.0.0 (Integromics). For the normalization of the data, although the expression of two endogenous controls was analyzed, inventors decided not to use them because they were not stable enough for normalization and inventors considered that it was better to normalize the data with the mean Ct value of all miRNA, as described by other groups [Mestdagh, P. at al. (2009). A novel and universal method for microRNA RT-qPCR data normalization. Genome Biol. 10, R64; Becker, C. et al. (2010). mRNA and microRNA quality control for RT-qPCR analysis. Methods 50, 237-243]. Because of general differences in gene expression patterns between men and women and based on the different prevalence of the disease between them, inventors thought that miRNA expression may also be different between the two groups. To test this hypothesis gender associated variability in the set of samples was analyzed. Inventors performed an analysis of equality of variances between men miRNA expression and women miRNA expression in each of the groups (relapse, remission, control and MS). One more category was included (Complete) in which all the samples were integrated. Thus, the data were divided into 160 subsets of values, 10 phenotype combination per 16 miRNAs.

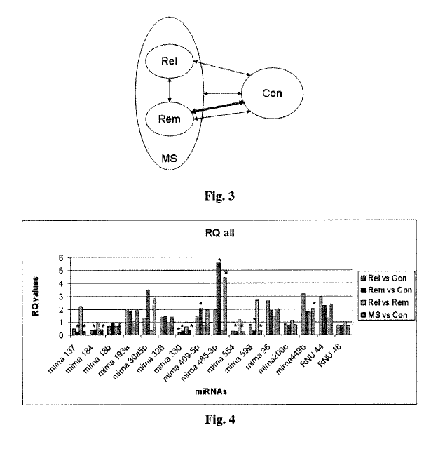

In the parametric analysis, several comparisons were performed using Limma t-test and p-value was corrected by the Benjamini-Hochberg algorithm (corrected p<0.05) (FIG. 3).

Apart from the classical data analysis inventors took a non-parametric approach, using DCT values, in order to find a miRNA or a subgroup of miRNA which might serve as biomarker for the disease. As the relapse group was smaller than the remission group, inventors only took into account the last one to have more statistical power. For this analysis a ranking method called symmetrical uncertainty (SU) was performed to measure the univariate relevance of each miRNA. This method sorts all the miRNAs according to their statistical relevance for the comparison made. The SU ranking is based on the mutual information between each miRNA expression level and the phenotype label. In order to gain robustness, inventors used a scheme of several runs coupled with a cross-validation split of the dataset. The data were split into k-folds computing the SU-metric for each feature in each fold independently and the average value is returned. This process was performed 10 times (10-times, 10-fold scheme) to compute average SU values. Although being a technical aspect, it is important to say that the seed of the cross validation split was different in each of the runs, so there were no equal divisions of the dataset.

Afterwards, inventors made an analysis to see if the studied miRNA were able to classify a given individual as control or remitting. To tackle this analysis the data using the equal width method dividing the expression levels into three intervals was discretized.

Different classifiers were applied to the dataset:
Bayesian networks: naïve Bayes, tree augmented naïve Bayes (TAN) and k-dependence Bayesian networks (kDB, k set at 3).
Decision trees: single trees and random forests of trees.
Logistic regression.
Distance based classifiers: k nearest neighbour algorithm with k set at 3.
Neural networks.

The evaluation of the classifiers was performed in terms of the accuracy (percentage of correctly classified individuals) and in terms of sensitivity and specificity (with respect to the remitting class). The estimations were obtained using a leave-one-out approach and a 10 times 10 fold cross-validations.

miRNA Targets

Target genes of miRNAs with a differential expression in any of the comparisons were searched using several programs available on-line: miRBase (release 14; September 2009), TargetScan 5.1, PicTar, DIANA microT, PITA. As each program uses different algorithm, inventors took the most conservative approach, taking into account only those predicted target genes which were common in all databases. With the gene lists obtained from this analysis, inventors performed a gene enrichment analysis using Panther to see which pathway, biological process or molecular function is overrepresented in each miRNA target gene list. In some cases, as the number of common mRNA targets predicted by all programs was very low, inventors only looked for the function of each gene one by one.

### 2. Results

In the present study the expression of 14 miRNAs using qPCR in 68 MS patients (15 in relapse and 53 in remission) and 36 controls who did not present any neurological symptoms was analyzed.

Looking to the gender associated variability analysis, only in 5 of the 80 comparisons of variances were found significant differences. Moreover, inventors analyzed whether, given a group of samples, the miRNA profile was similar between male and female in control and remitting groups. Inventors could see that there were no big differences in the general expression profiles between female/male controls and female/male remitting samples (data not shown). The mean values for each miRNA and for each gender were also collected and inventors could see that the mean values were very close one to each other. Just as a brief remark, inventors observed that the level of overlapping is higher within the remitting group comparing with controls.

It was concluded that there is not enough evidence on the data that suggests that the expression of these miRNAs is more stable in males than in females. According to this, inventors performed the rest of the analysis without taking into account the gender of the samples.

Comparing patients in remission with controls, in the parametric analysis, there were several miRNAs differentially expressed. Hsa-miR-409-5p (RQ=2.1) and hsa-miR-485-3p (RQ=5.6) were significantly overexpressed in the remission group while hsa-miR-137 (RQ=0.2), hsa-miR-184 (RQ=0.4), hsa-miR-330 (RQ=0.3), hsa-miR-554 (RQ=0.2) and hsa-miR-599 (RQ=0.3) were significantly underexpressed (Table 9).

TABLE 9
Summary of the relative quantification results obtained in multiple
comparisons with the parametric analysis, using Benjamini-Hochberg
corrected p-value (p < 0.05) and the non-parametric analysis.
Differentially expressed miRNAs are listed in different comparisons

| | | SU and |
|---|---|---|
| | Differentially | Classification |
| | expressed miRNAs | performance |
| Rel vs Con | miR-330 (0.21) | |
| (15 vs 36) | | |
| Rem vs Con | miR-409-5p (2.1) | miR-200c |
| (55 vs 36) | miR-485-3p (5.6) | miR-137 |
| | miR-137 (0.2) | miR-330 |
| | miR-184 (0.4) | miR-193a |
| | miR-330 (0.3) | |
| | miR-554 (0.2) | |
| | miR-599 (0.3) | |
| Rel vs Rem | — | |
| (15 vs 53) | | |
| MS vs Con | miR-485-3p (4.4) | |
| (68 vs 36) | miR-449b (2.1) | |
| | miR-137 (0.3) | |
| | miR-184 (0.4) | |
| | miR-330 (0.3) | |
| | miR-554 (0.2) | |
| | miR-599 (0.4) | |

Con: Control; MS: Multiple sclerosis; Rel: Relapse; Rem: Remission
[Numbers in brackets show the RQ value obtained in each case. In the last column results of the SU and classification performance analysis are shown only in the comparison performed. Only the statistically significant results are shown]

In the non-parametric approach, the symmetrical uncertainty (SU) analysis outputed 4 miRNAs (miR-200c, miR-330, miR-193a and miR-137) that significantly surpassed the rest, and so, it can be stated that these four miRNA are the most relevant features in the classification problem (FIG. 4).

In order to evaluate the potential of these miRNAs as biomarker of the disease, inventors discretized the data using the equal width method, dividing the expression levels into three intervals. Afterwards, 3 datasets according to the SU results were created:
1. All the studied miRNAs: 14 miRNAs.
2. miRNAs 200c, 96, 599, 554, 485-3p, 330, 193a and 137, discarding those miRNAs with the lowest correlation level with the class (very low average SU metric value): 8 miRNAs out of 14.
3. miRNAs with the highest correlation with the class (highest SU value): miR-200c, 330, 193a and 137): 4 miRNAs out of 14.

Later several classifiers of very different nature were applied to those 3 datasets: Bayesian network classifiers, decision trees, logistic regression, distance based classifiers and neural networks. The evaluation of the classifiers was made in terms of the accuracy, sensitivity and specificity (Table 10).

TABLE 10
Results of the different classifiers on the discretized dataset evaluated by means of
a leave-one-out (first 3 columns) and a repeated k-fold cross validation (10 times 10
fold, last 3 columns). The results show the accuracy, the sensitivity and the
specificity. The results have been computed with all the 14 miRNAs (first table)
and with 2 subsets: 200c, 96, 599, 554, 485-3p, 330, 193a, 137 (second table) and
200c, 330, 193a, 137 (last table). The best results obtained in each case appear
highlighted

| | Leave One Out | | | 10 times 10 fold cv | | |
|---|---|---|---|---|---|---|
| Classifier | Accuracy | Sensitivity | Specificity | Accuracy | Sensitivity | Specificity |
| | All | | | | | |
| Naive Bayes | 83.15 | 92.45 | 69.44 | 83.15 | 92.45 | 69.44 |
| TAN | 88.76 | 96.23 | 77.78 | 86.07 | 95.28 | 72.5 |
| 3-DB | 78.65 | 94.34 | 55.56 | 80.56 | 94.53 | 60 |
| Decission Trees | 83.15 | 90.57 | 72.22 | 81.69 | 88.11 | 72.22 |
| Logistic Regression | 82.02 | 88.68 | 72.22 | 79.1 | 85.85 | 69.17 |
| Random forest | 84.27 | 88.68 | 77.78 | 83.48 | 89.06 | 75.28 |
| 3 Nearest neighbour | 80.9 | 92.45 | 63.89 | 81.24 | 93.02 | 63.89 |
| Neural networks | 77.53 | 83.02 | 69.44 | 78.88 | 86.23 | 68.06 |
| | 200c, 96, 599, 554, 485.3p, 330, 193a, 137 | | | | | |
| Naive Bayes | 80.9 | 90.57 | 66.67 | 81.91 | 92.08 | 66.94 |
| TAN | 85.39 | 92.45 | 75 | 84.16 | 90.75 | 74.44 |
| 3-DB | 84.27 | 92.45 | 72.22 | 81.01 | 90 | 67.78 |
| Decission Trees | 86.52 | 92.45 | 77.78 | 84.61 | 90.94 | 75.28 |
| Logistic Regression | 76.4 | 83.02 | 66.67 | 77.41 | 85.28 | 65.83 |
| Random forest | 84.27 | 88.68 | 77.78 | 84.04 | 88.87 | 76.94 |
| 3 Nearest neighbour | 85.39 | 98.11 | 66.67 | 85.17 | 98.11 | 66.11 |
| Neural networks | 82.02 | 86.79 | 75 | 83.82 | 88.87 | 76.39 |
| | 200c, 330, 193a, 137 | | | | | |
| Naive Bayes | 84.27 | 96.23 | 66.67 | 84.27 | 96.23 | 66.67 |
| TAN | 84.27 | 88.68 | 77.78 | 84.49 | 90.19 | 76.11 |
| 3-DB | 79.78 | 84.91 | 72.22 | 79.33 | 85.09 | 70.83 |
| Decission Trees | 79.78 | 88.68 | 66.67 | 81.46 | 90.19 | 68.61 |
| Logistic Regression | 83.15 | 94.34 | 66.67 | 83.48 | 94.91 | 66.67 |
| Random forest | 84.27 | 88.68 | 77.78 | 83.48 | 89.62 | 74.44 |
| 3 Nearest neighbour | 79.78 | 90.57 | 63.87 | 78.88 | 91.51 | 60.28 |
| Neural networks | 83.15 | 88.68 | 75 | 82.47 | 89.06 | 72.78 |

In order to find differences in miRNA expression between relapse and remission moments of MS inventors also made the analysis comparing these two groups but they could not find any statistically significant results with the parametric analysis in none of the groups (Table 9). Comparing relapse group with the controls, it was found that hsa-miR-330 was significantly downregulated (RQ=0.21).

In the last comparison inventors took all patients without taking into account if they were in relapse or remission moment (MS). The results showed that there is a significant overexpession of hsa-miR-485-3p (RQ=4.4) and hsa-miR-449b (RQ=2.1) in patients while hsa-miR-137 (RQ=0.3), hsa-miR-184 (RQ=0.4), hsa-miR-330 (RQ=0.3), hsa-miR-554 (RQ=0.2) and hsa-miR-599 (RQ=0.4) were significantly underexpressed (Table 9).

Inventors searched for target genes of differentially expressed miRNAs as described in Material and Methods. With the gene lists obtained from this analysis, a gene enrichment analysis using Panther was performed to see which pathway, biological process or molecular function is overrepresented in each miRNA target gene list. In some cases, as the number of common mRNA targets predicted by all programs was very low, inventors only looked for the function of each gene one by one. This analysis did not gave any remarkable result.

### 3. Discussion

In the present work inventors have characterized the expression of 14 miRNAs selected from a previous study in a wider group of patients and controls with the objective of finding a candidate miRNA or group of miRNAs as biomarkers of the disease or relapse status. Finding a biomarker of the disease would be very helpful for diagnosis, due to the fact that nowadays the diagnosis is based on clinical, radiological and immunological criteria. It is considered that making an early diagnosis of MS is important to initiate the treatment as early as possible due to neurodegeneration processes that occur at the beginning of the disease. Apart from finding a disease biomarker it would be very interesting to find a relapse biomarker because, often, it is difficult to determine if the patient is suffering a relapse as the symptoms are subjective. Moreover, it is very common to suffer subclinical relapses, which do not present any clinical evidence.

Looking at the non-parametric analysis of symmetrical uncertainty we can state that there are 4 miRNAs (miR-200c, miR-330, miR-193a and miR-137) that are the most relevant for the classification problem. Looking at the classification performance results (Table 10), inventors observed that the predictions do not change too much when all the miRNAs are taken into account or only the miRNAs identified with de SU metric as the most relevant are used. This observation confirms that these miRNAs can be discarded without penalizing the classification task.

In general, it could be said that the promising results obtained in the evaluation of the performance of the classifiers, suggest that the expression of the miRNAs in the dataset have enough predicting power to distinguish between controls and remitting individuals, although it should be validated in a bigger set of samples.

Comparing the results obtained with the two approaches used in this study (parametric and non-parametric) it can be observed that there are some similarities. miR-330 and miR-137 (Table 9) are found to be deregulated in remission and they have shown to have enough predictive power, together with other two miRNAs, to distinguish the remission from the control group. This suggests that this two miRNAs might be good candidates for further studies. Moreover, miR-193a appears as important in remission in co-expression networks in the previous work and in the classification performance with the new dataset (Table 9). Therefore, although in the parametric analysis inventors did not find this miRNA significantly altered, it could be another candidate for future validations,

miR-330 is also underexpressed in relapse and in MS comparing with controls. Furthermore, it is not significantly downregulated if the two states of the disease are compared, suggesting that its differential expression is related to the disease.

In the present work inventors have analyzed a list of 14 miRNAs in which were included 6 of the 7 miRNAs reported on the previous study and comparing the results from both studies it could be said that miR-184 and miR-599 were validated as deregulated miRNAs in remission state of the disease (Table 9).

However, the direction of deregulation (to be up- or down-regulated) is not the same in both studies for these miRNA (Table 11). Whereas in the present study hsa-miR-184 and hsa-miR-599 are both downregulated in remission status comparing with controls, in the previous work were upregulated. These differences may be explained by the increase in the number of samples. Some of the differentially expressed miRNAs did not reach the statistic significance, probably by the reduced number of samples.

TABLE 11
Comparison of the statistically significant results obtained in the present study
with different methods and in the previous study

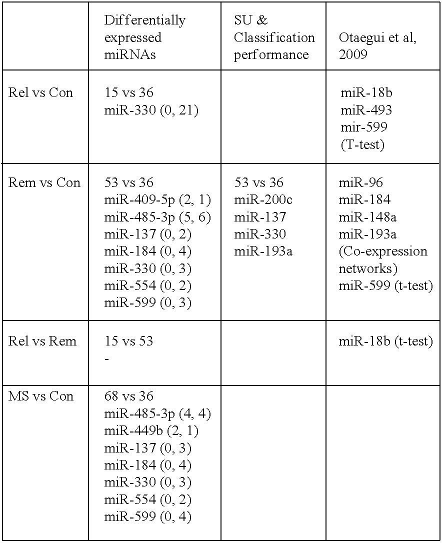

Con: Control;
MS: Multiple sclerosis;
Rel: Relapse;
Rem: Remission
[Boxes in grey mean that comparison has not been performed]

In the last months another three works have studied the expression of miRNAs in multiple sclerosis patients. Keller and colleagues [Keller, A. et al. (2009). Multiple sclerosis: microRNA expression profiles accurately differentiate patients with relapsing-remitting disease from healthy controls. PLoS. ONE. 4, e7440] analysed the expression of 866 miRNAs in blood cells of 20 MS patients and 19 healthy controls. They found 145 deregulated miRNAs in MS patients. Among these 145 miRNAs, 7 were found to be deregulated also in our group's previous work. In the present work there is only one overlapping miRNA, hsa-miR-330, among 145 deregulated miRNAs found by Keller and colleagues. This miRNA is found underexpressed in both studies in patients comparing with controls (Table 12).

The second article relating miRNAs to MS directly, analyses the expression of miRNAs isolated from active and inactive MS lesions in the white matter of patients and controls (people without any known neurological disease) [Junker, A. et al. (2009). MicroRNA profiling of multiple sclerosis lesions identifies modulators of the regulatory protein CD47. Brain 132, 3342-3352]. They studied miRNA expression profile of active and inactive MS lesions and found different deregulated miRNAs in each case. There are 6 miRNAs studied in the present work that have been found deregulated in MS lesions by Junker and colleagues: hsa-miR-184, hsa-miR-193a, hsa-miR-200c, hsa-miR-30a-5p, hsa-miR-328 and hsa-miR-330. Hsa-miR-30a-5p and hsa-miR-328 have not been found significantly deregulated, but among the other four are some coincidences between the two studies. For instance, Junker and colleagues also found hsa-miR-330 downregulated in inactive MS lesions and the present results herein indicate that in remission status this miRNA is also downregulated. However, in the present study inventors have also found this miRNA downregulated in relapse and Junker and colleagues have not found this miRNA expression altered in active lesions. Moreover, miR-193a, miR-200c and miR-30a-5p that have been found altered in MS lesions, have not been found differentially expressed in blood of MS patients. Inventors are conscious that the fact that the tissue analyzed in both studies is different makes this comparison difficult, because it cannot be known which findings are due to the disease or due to the different tissue.

The last work studying miRNA expression in MS patients analysed miRNA expression in T and B cells, separately [Lindberg, R. L. et al. (2010). Altered expression of miR-17-5p in CD4(+) lymphocytes of relapsing-remitting multiple sclerosis patients. Eur. J. Immunol. 40, 888-898]. First, they made miRNA arrays and then validated the results with qPCR. The most remarkable result, comparing with these results, is that in CD4 T cells, they found miR-485-3p upregulated and inventors have also found this miRNA upregulated in both the remission and all MS patients comparing with controls (Table 12). This observation suggests that the deregulation of miR-485-3p observed by inventors could come specifically from T cells.

The results of the different studies suggest that the miR-330 might play an important role in the pathogenesis, due to the fact that has been found deregulated in three groups of patients, with different analysis both in blood and brain tissues.

Apart from the different results among these studies, the fact that miRNA are involved in multiple sclerosis is more evident. Either their altered expression takes part in the pathogenesis or is a consequence of different phenomena occurring during the evolution of the disease. Furthermore, miRNAs could be new therapeutic targets, in case it is demonstrated they take part in pathogenesis, because they participate in gene regulation. Increasing the knowledge about the biological role of miRNAs will allow exploring the possibility of new therapeutic targets. Developing RNA-based therapies could help slowing down the progression of the diseases although there is an unknown field that has to be explored in the future.

According to the data provided by this invention, it can be concluded that miRNAs might be good candidates for disease biomarkers, as inventors have reached good values of accuracy, sensitivity and sensibility to distinguish a MS patient from a healthy individual. Another interesting issue is to understand the biological role of deregulated miRNA in the disease, and so, further studies are needed. The need of some criteria to have more homogeneous groups among the MS patients is appreciated, due to the fact that the heterogeneity of the disease may mask the differences in miRNA expression.

TABLE 12
Results obtained in other studies are shown comparing with the present study. The tendency of the deregulation is shown in each
case (up-arrow: upregulation. Down-arrow: downregulation, two-head arrow: no deregulation. Star next to the arrow means that
it is statistically significant. There are cases in which the miRNA is not detected or studied in other works. Next to the authors
name, the studied tissue or cell type is specified

| | Invention | | | | Otaegul (2009) | | | Keller | Lindberg |
|---|---|---|---|---|---|---|---|---|---|
| | (blood) | | | | (blood) | | | (blood) | (CD4) |
| | Comparison | | | | | | | | |
| miRNA | Rel vs Con | Rem vs Con | Rel vs Rem | MS vs Con | Rel vs Con | Rem vs Con | Rel vs Rem | MS vs Con | MS vs Con |
| hsa-miR-184 | ⇓ | ⇓* | [symbol] | ⇓* | ⇑ | ⇑ | ⇑ | ⇓ | ~ |
| hsa-miR-18b | ⇓ | [symbol] | ⇓ | [symbol] | ⇑* | ⇑ | ⇑* | ⇓ | ~ |
| hsa-miR-193a | ⇑ | ⇑ | [symbol] | ⇑ | ⇓ | [symbol] | ⇓ | ~ | ⇑* |
| hsa-miR-200c | ⇓ | ⇓ | ⇑ | ⇓ | ⇑ | ⇑ | ⇑ | ⇑ | ~ |
| hsa-miR-30a-5p | ⇑ | ⇑ | ⇓ | ⇑ | [symbol] | [symbol] | [symbol] | ~ | ~ |
| hsa-miR-328 | ⇑ | ⇑ | [symbol] | ⇑ | ⇑ | ⇓* | ⇑* | ⇑ | ~ |
| hsa-miR-330 | ⇓* | ⇓* | ⇓ | ⇓* | ⇑* | [symbol] | ⇑ | ⇓* | ~ |
| hsa-miR-409-5p | ⇑ | ⇑* | ⇓ | ⇑ | ⇑ | ⇓ | ⇑* | ⇑ | ~ |
| hsa-miR-449b | ⇑ | ⇑ | ⇑ | ⇑* | ⇑ | ⇑* | ⇓ | ⇑ | ~ |
| hsa-miR-485-3p | ⇑ | ⇑* | ⇓ | ⇑* | ⇑ | ⇑ | ⇑ | ⇓ | ⇑* |
| hsa-miR-554 | ⇓ | ⇓* | ⇑ | ⇓* | ⇑ | ⇓ | ⇑ | ⇓ | ~ |
| hsa-miR-599 | ⇓ | ⇓* | ⇑ | ⇓* | ⇑* | ⇑ | ⇑ | ⇓ | ~ |
| hsa-miR-96 | ⇑ | ⇑ | ⇑ | ⇑ | ⇑ | ⇑* | ⇓ | ⇑ | ~ |
| hsa-miR-137 | ⇓ | ⇓* | ⇑ | ⇓* | ⇑ | [symbol] | ⇑* | ⇓ | ~ |

1. A method of diagnosing Multiple Sclerosis (MS) in a subject, which comprises comparing:
a) the level of expression of a miRNA selected from the group consisting of hsa-mir-18b, hsa-mir-493, hsa-mir-599, hsa-mir-96, hsa-mir-148a, hsa-mir-184, hsa-mir-193, hsa-mir-193a, hsa-mir-328, hsa-mir-409-5p, hsa-mir-449b, hsa-mir-485-3p, hsa-mir-200c, hsa-mir-330, hsa-mir-554, and combinations thereof, in a sample from said subject; and
b) the normal level of expression of said miRNA(s) in a reference sample,
wherein a statistically significant deregulation in the level of expression of said miRNA hsa-mir-18b, hsa-mir-493, hsa-mir-599, hsa-mir-96, hsa-mir-148a, hsa-mir-184, hsa-mir-193, hsa-mir-193a, hsa-mir-409-5p, hsa-mir-449b, hsa-mir-485-3p, hsa-mir-554, in the subject sample with respect to the normal level of said miRNA(s) in the reference sample is indicative that the subject is afflicted with MS,
wherein a statistically significant increase in the level of expression of hsa-mir-328 in the subject sample with respect to the normal level of said miRNA(s) in the reference sample is indicative that the subject is afflicted with MS,
wherein a statistically significant decrease in the level of expression of hsa-mir-330 in the subject sample with respect to the normal level of said miRNA(s) in the reference sample is indicative that the subject is afflicted with MS, and
wherein a statistically significant decrease in the level of expression of hsa-mir-200c in the subject sample with respect to the normal level of said miRNA(s) in the reference sample is indicative that the subject is afflicted with MS,
or alternatively, comparing
a′) the level of expression of a set of miRNA comprising hsa-mir-137, hsa-mir-554, hsa-mir-449b, hsa-mir-30a-5p, hsa-mir-96, hsa-mir-599, hsa-mir-200c, hsa-mir-409-5p, hsa-mir-485-3p, hsa-mir-164, hsa-mir-328, hsa-mir-193a, hsa-mir-330 and hsa-mir-18b, in a sample from said subject; and
b′) the normal level of expression of said miRNAs in a reference sample,
wherein a statistically significant deregulation in the level of expression of said miRNAs comprising hsa-mir-137, hsa-mir-554, hsa-mir-449b, hsa-mir-30a-5p, hsa-mir-96, hsa-mir-599, hsa-mir-200c, hsa-mir-409-5p, hsa-mir-485-3p, hsa-mir-184, hsa-mir-328, hsa-mir-193a, hsa-mir-330 and hsa-mir-18b, in the subject sample with respect to the normal level of said miRNAs in the reference sample is indicative that the subject is afflicted with MS;
or alternatively, comparing
a″) the level of expression of a set of miRNA comprising hsa-mir-599, hsa-mir-96, hsa-mir-193a, hsa-mir-485-3p, hsa-mir-200c, hsa-mir-330, hsa-mir-554 and hsa-miR-137, in a sample from said subject; and
b″) the normal level of expression of said miRNAs in a reference sample,
wherein a statistically significant deregulation in the level of expression of said miRNAs hsa-mir-599, hsa-mir-96, hsa-mir-193a, hsa-mir-485-3p, hsa-mir-200c, hsa-mir-330, hsa-mir-554 and hsa-mir-137, in the subject sample with respect to the normal level of said miRNAs in the reference sample is indicative that the subject is afflicted with MS;
or alternatively, comparing
a′″) the level of expression of a set of miRNAs comprising hsa-mir-193a, hsa-mir-200c, hsa-mir-330 and hsa-miR-137, in a sample from said subject; and
b′″) the normal level of expression of said miRNAs in a reference sample,
wherein a statistically significant deregulation in the level of expression of said miRNAs hsa-mir-193a, hsa-mir-200c, hsa-mir-330 and hsa-mir-137, in the subject sample with respect to the normal level of said miRNAs in the reference sample is indicative that the subject is afflicted with MS.

2. A method according to claim 1, which comprises comparing:
a) the level of expression of said miRNA hsa-mir-18b, hsa-mir-493, hsa-mir-599, hsa-mir-96, hsa-mir-148a, hsa-mir-184, hsa-mir-193, hsa-mir-193a, hsa-mir-328, hsa-mir-409-5p, hsa-mir-449b or hsa-mir-485-3p, hsa-mir-200c, hsa-mir-330 and hsa-mir-554, in a sample from said subject; and
b) the normal level of expression of said miRNA(s) in a reference sample,
wherein a statistically significant deregulation in the level of expression of said miRNAs in the subject sample with respect to the normal level of said miRNA(s) in the reference sample is indicative that the subject is afflicted with MS.

3. Method according to claim 1, wherein a statistically significant increase in the level of expression of said miRNA hsa-mir-18b, hsa-mir-493, hsa-mir-96, hsa-mir-148a, hsa-mir-193, hsa-mir-193a, hsa-mir-409-5p, hsa-mir-449b, or hsa-mir-485-3p, in the subject sample with respect to the normal level of said miRNA(s) in the reference sample is indicative that the subject is afflicted with MS, or, wherein a statistically significant decrease in the level of expression of said miRNA(s) hsa-mir-599, hsa-mir-184 or hsa-mir-554 in the subject sample with respect to the normal level of said miRNA(s) in the reference sample is indicative that the subject is afflicted with MS.

4.-6. (canceled)

7. The method according to claim 1, wherein a statistically significant increase in the level of expression of said miRNA, hsa-mir-449b, hsa-mir-30a-5p, hsa-mir-96, hsa-mir-409-5p, hsa-mir-485-3p, hsa-mir-32B and/or hsa-mir-193a, in the subject sample with respect to the normal level of said miRNA(s) in the reference sample is indicative that the subject is afflicted with MS, or wherein a statistically significant decrease in the level of expression of said miRNA hsa-mir-137, hsa-mir-554, hsa-mir-599, hsa-mir-200c, hsa-mir-184 and/or hsa-mir-330 in the subject sample with respect to the normal level of said miRNA(s) in the reference sample is indicative that the subject is afflicted with MS.

8. A method for assessing if a subject afflicted with Multiple Sclerosis (MS) is experiencing a relapse, which comprises comparing:
a) the level of expression of a miRNA selected from the group consisting of hsa-mir-493, hsa-mir-193, hsa-mir-193a, hsa-mir-328, hsa-mir-409-5p, hsa-mir-449b, hsa-mir-485-3p, hsa-mir-96 and combinations thereof, in a test sample from said subject; and
b) the normal level of expression of said miRNA(s) in a reference sample,
wherein a statistically significant increase in the level of expression of said miRNA(s) in the subject sample with respect to the normal level of said miRNA(s) in the reference sample is indicative that the subject afflicted with MS is experiencing a relapse;
or alternatively, comparing
a′) the level of expression of a miRNA selected from the group consisting of hsa-mir-96, hsa-mir-148a, hsa-mir-184, hsa-mir-193, hsa-mir-18b, hsa-mir-599, hsa-mir-200c, hsa-mir-330, hsa-mir-554, hsa-mir-137 and combinations thereof, in a test sample from said subject; and
b′) the level of expression of said miRNA(s) in a control smile, said control sample being a sample from the same subject under analysis obtained during a period in which said subject afflicted with MS under analysis is experiencing a remission,
wherein a statistically significant decrease in the level of expression of said miRNA(s) in the subject sample with respect to the level of said miRNA(s) in the control sample is indicative that the subject afflicted with MS is experiencing a relapse.

9. A method for assessing if a subject afflicted with Multiple Sclerosis (MS) is experiencing a remission, which comprises comparing:
a) the level of expression of a miRNA selected from the group consisting of hsa-mir-96, hsa-mir-148a, hsa-mir-193, hsa-mir-193a, hsa-mir-328, hsa-mir-409-5p, hsa-mir-449b, hsa-mir-485-3p, and combinations thereof, in a test sample from said subject; and
b) the normal level of expression of said miRNA(s) in a reference sample,
wherein a statistically significant increase in the level of expression of said miRNA(s) in the subject sample with respect to the normal level of said miRNA(s) in the reference sample is indicative that the subject afflicted with MS is experiencing a remission;
or alternatively, comparing:
a) the level of expression of a miRNA selected from the group consisting of hsa-mir-18b, hsa-mir-493, hsa-mir-599, and combinations thereof, in a test sample from said subject; and
b) the level of expression of said miRNA(s) in a control sample, said control sample being a sample from the same subject under analysis obtained during a period in which said subject afflicted with MS under analysis is experiencing a relapse,
wherein a statistically significant decrease in the level of expression of said miRNA(s) in the subject sample with respect to the level of said miRNA(s) in the control sample is indicative that the subject afflicted with MS is experiencing a remission.

10-11. (canceled)

12. The method according to claim 1, wherein said sample comprises blood mononuclear cells.

13. The method according to claim 1, wherein the level of expression of said miRNAs is determined by multiplex and/or singleplex real-time RT-PCR; or by single-molecule detection; or by a bead-based flow cytometric method; or by an assays using arrays of nucleic acids.

14. The method according to claim 13, wherein the level of expression of said miRNAs is determined by real-time quantitative RT-PCR (gRT-PCT).

15. The method according to claim 1, wherein said miRNA is selected from the group consisting of hsa-mir-18b, hsa-mir-96, hsa-mir-148a, hsa-mir-184, hsa-mir-493, hsa-mir-599, hsa-mir-193, hsa-mir-193a, hsa-mir-200c, hsa-mir-330 and combinations thereof.

15. (canceled)

16. A method of designing a therapy for a subject afflicted with Multiple Sclerosis (MS), which comprises:
determining if a subject afflicted with MS is experiencing a relapse according to the method of claim 8, and selecting a drug suitable for treatment of MS in a relapse status; or, alternatively,
determining if a subject afflicted with MS is experiencing a remission according to the method of claim 9, and selecting a drug suitable for treatment of MS in a remission status.

17. The method according to claim 2, wherein a statistically significant increase in the level of expression of said miRNA hsa-mir-18b, hsa-mir-493, hsa-mir-96, hsa-mir-148a, hsa-mir-193, hsa-mir-193a, hsa-mir-409-5p, hsa-mir-449b or hsa-mir-485-3p, in the subject sample with respect to the normal level of said miRNA(s) in the reference sample is indicative that the subject is afflicted with MS, or wherein a statistically significant decrease in the level of expression of said miRNA hsa-mir-599, hsa-mir-184 or hsa-mir-554 in the subject sample with respect to the normal level of said miRNA(s) in the reference sample is indicative that the subject is afflicted with MS.

18. The method according to claim 8, wherein said sample comprises blood mononuclear cells.

19. The method according to claim 9, wherein said sample comprises blood mononuclear cells.

20. The method according to claim 8, wherein the level of expression of said miRNAs is determined by multiplex and/or singleplex real-time RT-PCR; or by single-molecule detection; or by a bead-based flow cytometric method; or by an assays using arrays of nucleic acids.

21. The method according to claim 9, wherein the level of expression of said miRNAs is determined by multiplex and/or singleplex real-time RT-PCR; or by single-molecule detection; or by a bead-based flow cytometric method; or by an assays using arrays of nucleic acids.

22. The method according to claim 20, wherein the level of expression of said miRNAs is determined by real-time quantitative RT-PCR (qRT-PCT).

23. The method according to claim 21, wherein the level of expression of said miRNAs is determined by real-time quantitative RT-PCR (qRT-PCT).

24. The method according to claim 1, wherein said miRNA is selected from the group consisting of hsa-mir-193a, hsa-mir-200c, hsa-mir-330 and combinations thereof.


SEQUENCE LISTING (17 sequences)

SEQ ID NO: 1 - RNA, 22 nt, Homo sapiens
uaaggugcaucuagugcaguua

SEQ ID NO: 2 - RNA, 22 nt, Homo sapiens
uuguacaugguaggcuuucauu

SEQ ID NO: 3 - RNA, 20 nt, Homo sapiens
guugugucaguuuaucaaac

SEQ ID NO: 4 - RNA, 22 nt, Homo sapiens
uuuggcacuagcacauuuuugc

SEQ ID NO: 5 - RNA, 22 nt, Homo sapiens
ucagugcacuacagaacuuugu

SEQ ID NO: 6 - RNA, 22 nt, Homo sapiens
uggacggagaacugauaagggu

SEQ ID NO: 7 - RNA, 22 nt, Homo sapiens
uguaaacauccucgacuggaag

SEQ ID NO: 8 - RNA, 21 nt, Homo sapiens
aacuggccuacaaagucccag

SEQ ID NO: 9 - RNA, 22 nt, Homo sapiens
aacuggccuacaaagucccagu

SEQ ID NO: 10 - RNA, 23 nt, Homo sapiens
uaauacugccggguaaugaugga

SEQ ID NO: 11 - RNA, 22 nt, Homo sapiens
cuggcccucucugcccuuccgu

SEQ ID NO: 12 - RNA, 23 nt, Homo sapiens
gcaaagcacacggccugcagaga

SEQ ID NO: 13 - RNA, 23 nt, Homo sapiens
agguuacccgagcaacuuugcau

SEQ ID NO: 14 - RNA, 22 nt, Homo sapiens
aggcaguguauuguuagcuggc

SEQ ID NO: 15 - RNA, 22 nt, Homo sapiens
gucauacacggcucuccucucu

SEQ ID NO: 16 - RNA, 21 nt, Homo sapiens
gcuaguccugacucagccagu

SEQ ID NO: 17 - RNA, 23 nt, Homo sapiens
uuauugcuuaagaauacgcguag